(19) United States
(12) Patent Application Publication
(10) Pub. No.: US 2024/0199762 A1
(43) Pub. Date: Jun. 20, 2024

(54) ANTI-FIBROBLAST ACTIVATION PROTEIN ANTIBODIES

(71) Applicant: PHILOGEN S.P.A, Siena (IT)

(72) Inventors: Roberto DE LUCA, Otelfingen (CH); Lisa NADAL, Otelfingen (CH); Frederik PEISSERT, Otelfingen (CH)

(73) Assignee: PHILOGEN S.P.A, Siena (IT)

(21) Appl. No.: 18/555,189

(22) PCT Filed: Apr. 22, 2022
(86) PCT No.: PCT/EP2022/060776

§ 371 Date: Oct. 12, 2023

(30) Foreign Application Priority Data
Apr. 23, 2021 (EP) 21170280.8
Aug. 30, 2021 (EP) 21193852.7
Jan. 4, 2022 (EP) 22150197.6
Mar. 2, 2022 (EP) 22159821.2

(51) Int. Cl. C07K 16/40; A61K 39/395; A61K 45/06; A61K 47/68; A61K 51/10; A61P 35/00

(52) U.S. Cl. CPC C07K 16/40; A61K 39/3955; A61K 45/06; A61K 47/6813; A61K 47/6871; A61K 51/1075; A61P 35/00; C07K 2317/626; C07K 2317/94

(57) ABSTRACT

The application relates to the diagnosis and treatment of diseases, including cancer, autoimmune diseases and inflammatory disorders. The invention provides, and involves the use of, antibody molecules that bind fibroblast activation protein (FAP) from humans, sheep, pigs and domestic dogs. The antibody molecules may be conjugated to a pro-inflammatory agent, an anti-inflammatory agent, a biocidal molecule, a cytotoxic molecule, or a radioisotope.

### FIELD OF THE INVENTION

The present invention relates to the diagnosis and treatment of diseases, including cancer, autoimmune diseases and inflammatory disorders. The invention provides, and involves the use of, antibody molecules that bind fibroblast activation protein (FAP) from humans, sheep, pigs and domestic dogs. The antibody molecules may be conjugated to a pro-inflammatory agent, an anti-inflammatory agent, a biocidal molecule, a cytotoxic molecule, or a radioisotope.

### BACKGROUND

Fibroblast activation protein (FAP) is a 95-kDa, cell surface-bound, type II transmembrane glycoprotein and belongs to the family of serine prolyl oligopeptidases. Expression of FAP in the majority of adult human tissues is rare but FAP is known to be upregulated in a wide variety of human cancers, as well as in several inflammatory and autoimmune diseases. Specifically, FAP has been shown to be expressed in rheumatoid myofibroblast-like synoviocytes in patients with rheumatoid arthritis. In the cancer setting, FAP has been shown to be expressed in tumour stroma and on tumour-associated fibroblasts. Importantly, FAP expression has been reported in over 90% of all human carcinomas and stromal fibroblasts are known to play an important part in the development, growth and metastasis of carcinomas. FAP has therefore been suggested as a promising target for anti-cancer therapy, as well as inflammatory and autoimmune disorders associated with FAP expression.

A number of antibodies which bind human FAP have been described in the art, including by the present applicant in WO2016/116399. In addition, several FAP inhibitors are currently being investigated as cancer therapeutics, including several anti-FAP antibodies. However, no FAP inhibitors are currently approved for the treatment of cancer or the treatment of inflammatory or autoimmune diseases, such as rheumatoid arthritis, in human patients. Thus, there remains a need in the art for further antibody-based FAP inhibitors.

The present invention has been devised in light of the above considerations.

### SUMMARY OF THE INVENTION

The present inventors have isolated high affinity antibody molecules which bind human Fibroblast Activation Protein (FAP), as well as FAP from domestic dogs (Canis familiaris; referred to as “canine” FAP herein), sheep (referred to as “ovine” FAP herein) and pigs (referred to as “porcine” FAP herein) with high affinity. This is in contrast to a number of known anti-FAP antibodies for which no cross-reactivity with canine, ovine and porcine FAP was observed (Examples 5 and 8).

Cross-reactivity with canine, ovine and porcine FAP opens up new avenues for evaluating the efficacy and tolerability of the anti-FAP antibody molecules, as well as their application. Large animals such as sheep and pigs represent useful animal models for the evaluation of anti-FAP antibodies, as results are expected to be more representative of human patients, than those obtained using smaller animal models, such as, e.g. mice [Kuyinu et al. J Orthopaedic Surgery & Research (2016) 11:19]. For example, pigs provide suitable animal models for the study of the pathogenic mechanisms which may be operative in rheumatoid arthritis. In fact, erysipelothrix arthritis can be induced in pigs by a single dose administration of Erysipelothrix Rusopathiae which produces joint lesions [RA Drew, Proc. Roy. Soc. Medicine, 994-997, Vol. 65]. Sheep are also routinely used in cartilage repair studies due to their similarities to humans, and in particular between ovine and human mesenchymal stem/stromal cell (MSC's) [Music et al., Osteoarthritis and Cartilage 26 (2018) 730-740].

In addition, the domestic dog, represents a useful animal model to assess cancer therapeutics. Naturally occurring cancers in domestic dogs are progressively leveraged as a valuable source of information due to their closely related pathophysiology to human cancers. In fact, even at the molecular level, canine carcinomas reflect the genomic aberrations found in human cancers [Ettlin et al., Int J Mol Sci, 18, 1101 (2017)]. For example, 90% of canine mast cell tumors have been shown to express FAP in the stroma [Giuliano et al., J Comp Path, 156, 14-20 (2017)]. In conclusion dogs, sheep and pigs represent promising animal models for translational studies to determine the efficacy of anti-FAP therapeutics in the treatment of cancer and of various types of inflammatory or autoimmune diseases, such as arthritis.

The anti-FAP antibody molecules of the present invention are also expected to find application in the treatment of cancer in domestic dogs.

In a first aspect, the present invention thus provides an antibody molecule that binds human, ovine, porcine and canine FAP, preferably the extracellular domain of human, ovine, porcine and canine FAP. The sequence of the extracellular domain of human FAP (hFAP) is shown in SEQ ID NO: 1, the sequence of the extracellular domain of canine FAP (caFAP) is shown in SEQ ID NO: 2, the sequence of the extracellular domain of ovine FAP (oFAP) is shown in SEQ ID NO: 72 and the sequence of the extracellular domain of porcine FAP (pFAP) is shown in SEQ ID NO: 73.

The antibody molecule preferably comprises the HCDR1, HCDR2, and HCDR3 sequences of the “7NP2” antibody set forth in SEQ ID NOs 3, 4 and 5, respectively, and/or the LCDR1, LCDR2 and LCDR3 sequences of the 7NP2 antibody set forth in SEQ ID NOs 6, 7 and 8, respectively. An antibody which comprises these 6 CDR sequences has been shown to be capable of binding the extracellular domain of human, canine, ovine and porcine FAP.

In a preferred embodiment, the antibody molecule comprises the VH domain or VL domain sequence, but preferably the VH domain and VL domain sequence, of the 7NP2 antibody molecule set forth in SEQ ID NOs 9 and 10, respectively.

An antibody molecule, as referred to herein, may be in any suitable format. Many antibody molecule formats are known in the art and include both complete antibody molecule molecules, such as IgG, as well as antibody fragments, such as a single chain Fv (scFv), diabodies, or single-chain diabodies. The term “antibody molecule” as used herein encompasses both complete antibody molecule molecules and fragments of antibody molecules, in particular antigen-binding fragments. In a preferred embodiment, the antibody molecule consists of or comprises an scFv, a small immunoprotein (SIP), a diabody, a single-chain diabody, or a (complete) IgG molecule, such as an IgG1 or IgG4 molecule.

Inhibition of FAP has been proposed as a mechanism for countering cartilage degradation in rheumatoid arthritis, an autoimmune and inflammatory disease associated angiogenesis [Waldele et al., Deficiency of fibroblast activation protein alpha ameliorates cartilage destruction in inflammatory destructive arthritis, Arthritis Res. Ther., 17(1): 12 (2015)]. The antibody molecule of the invention may thus find application in the treatment of diseases, such as inflammatory and autoimmune diseases like rheumatoid arthritis, through inhibition of FAP even in the absence of a therapeutic agent conjugated to the antibody molecule.

In addition, FAP has been shown to be useful as a marker for cancers, as well as inflammatory and autoimmune diseases and disorders, localising to sites of disease with high specificity. The antibody of the invention may thus be employed in the imaging, detection and diagnosis, of diseases and disorders characterised, or associated with, the expression of FAP. In this context, the antibody molecule may be used as is and later detected using e.g. a secondary antibody molecule or may be conjugated to a detectable label. An antibody molecule of the present invention may thus be used as is, i.e. in unconjugated form, or may be conjugated to a therapeutic or diagnostic agent to provide a conjugate, but preferably is used in the form of a conjugate. The choice of agent conjugated to the antibody molecule will depend on the intended application of the conjugate. For example, where the conjugate is intended for the treatment of a disease or disorder, the conjugate may comprise an antibody molecule of the invention and a pro-inflammatory agent, an anti-inflammatory agent, a biocidal molecule, a cytotoxic molecule, a radioisotope, a photosensitizer, an enzyme, a hormone, or an immunosuppressive agent. Where the conjugate is intended for use in imaging, detecting, or diagnosing a disease or disorder, the conjugate may comprise an antibody molecule of the invention and a detectable label, such as a radioisotope, e.g. a non-therapeutic radioisotope. Depending on the agent conjugated to the antibody molecule, the conjugate may be or may comprise a single-chain protein. Where the conjugate is a single-chain protein, the entire protein can be expressed as a single polypeptide or fusion protein. In this case, the agent may be conjugated to the antibody molecule by means of a peptide linker. Fusion proteins have the advantage of being easier to produce and purify since they consist of a single species. This facilitates production of clinical-grade material. Alternatively, the agent may be conjugated to the antibody molecule by means of a cleavable linker.

The invention also provides isolated nucleic acids encoding the antibody molecules and conjugates of the invention. The skilled person would have no difficulty in preparing such nucleic acids using methods well-known in the art. An isolated nucleic acid may be used to express the antibody molecule or conjugate of the invention, for example by expression in a bacterial, yeast, insect or mammalian host cell. A preferred host cell is E. coli. The nucleic acid will generally be provided in the form of a recombinant expression vector for expression. Host cells in vitro comprising such nucleic acids and expression vectors are part of the present invention, as is their use for expressing the antibody molecules and conjugates of the invention, which may subsequently be purified from cell culture and optionally further formulated into a pharmaceutical composition.

An antibody molecule or conjugate of the invention may be provided for example in a pharmaceutical composition, and may be employed for medical use as described herein, either alone or in combination with one or more further therapeutic agents, such as a Janus kinase (JAK) inhibitor or an immunomodulatory agent such as an anti-PD-1 antibody. Alternatively, the antibody molecule or conjugate of the invention may be provided in a diagnostic composition and may be employed for diagnostic use as described herein.

The present invention thus also relates to an antibody molecule or conjugate of the invention for use in a method for treatment of the human or animal body by therapy. For example, an antibody molecule or conjugate of the invention may be for use in a method of treating an inflammatory disorder, inhibiting angiogenesis, treating cancer, and/or treating an autoimmune disease in a patient. The invention also relates to a method of treating an inflammatory disorder, inhibiting angiogenesis, treating cancer, and/or treating an autoimmune disease in a patient, the method comprising administering a therapeutically effective amount of an antibody molecule or conjugate of the invention to the patient. The use of an antibody molecule or conjugate of the invention for the manufacture of a medicament for the treatment of an inflammatory disorder, inhibiting angiogenesis, treating cancer, and/or treating an autoimmune disease, is also contemplated. An inflammatory disorder or autoimmune disease, as referred to herein, may be rheumatoid arthritis, ostheoarthritis or inflammatory bowel disease, such Crohn's disease or ulcerative colitis. The present invention further relates to an antibody molecule of the invention for use in a method of delivering a molecule to sites of an inflammatory disorder, sites of neovasculature which are the result of angiogenesis, sites of cancer and/or sites of autoimmune disease in a patient. The invention also relates to a method of delivering a molecule to sites of an inflammatory disorder, sites of neovasculature which are the result of angiogenesis, sites of cancer and/or sites of autoimmune disease in a patient comprising administering to the patient an antibody molecule of the invention, wherein the antibody molecule is conjugated to the molecule.

A further aspect of the invention relates to an antibody molecule or conjugate of the invention for use in a method of imaging, detecting, or diagnosing an inflammatory disorder, angiogenesis, cancer, and/or an autoimmune disease in a patient. The invention also relates to a method of imaging, detecting, or diagnosing an inflammatory disorder, angiogenesis, cancer, and/or an autoimmune disease in a patient comprising administering an antibody molecule or conjugate of the invention to the patient. The method may be an in vitro or an in vivo method. Also encompassed within the scope of the invention is the use of an antibody molecule or conjugate of the invention for the manufacture of a diagnostic product for imaging, detecting, or diagnosing an inflammatory disorder, angiogenesis, cancer, and/or an autoimmune disease.

A patient, as referred to herein, is preferably a human patient. Alternatively, the patient may be a domestic dog (Canis familiaris).

The invention includes the combination of the aspects and preferred features described except where such a combination is clearly impermissible or expressly avoided.

SUMMARY OF THE FIGURES

Embodiments and experiments illustrating the principles of the invention will now be discussed with reference to the accompanying figures in which:

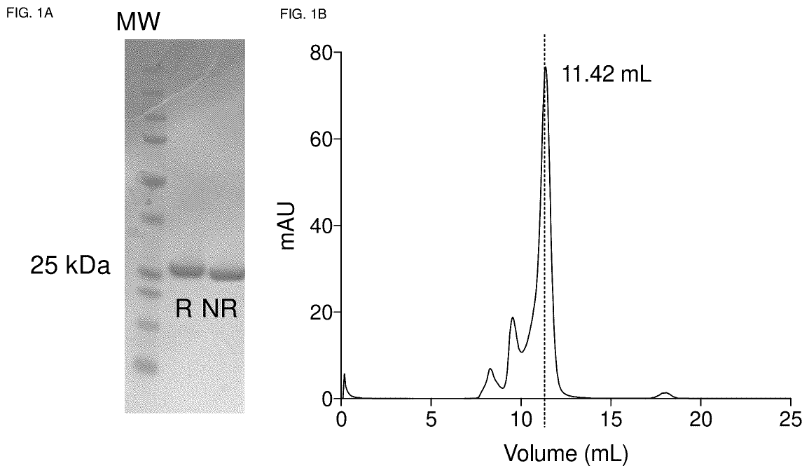

FIG. 1 shows the characterization of anti-FAP scFv(C5). FIG. 1A shows the results of SDS-PAGE analysis of scFv(C5). The scFv had the expected size of 25 kDa under non-reducing (NR) and reducing (R) conditions, respectively. FIG. 1B shows the results of size exclusion chromatogram of scFv(C5). The monomeric form of the scFv was eluted from the column at 11.4 mL.

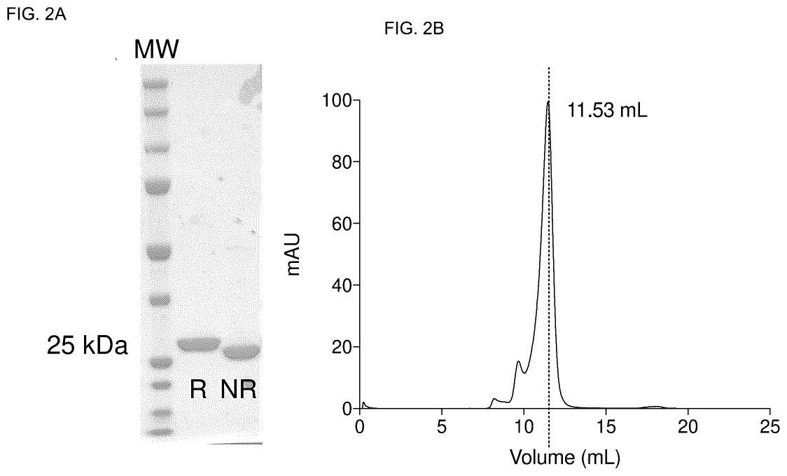

FIG. 2 shows the characterization of the affinity matured anti-FAP scFv(7NP2). FIG. 2A shows the results of SDS-PAGE analysis of scFv(7NP2). The protein had the expected size of 25 kDa under non-reducing and reducing conditions, respectively. FIG. 2B shows a size exclusion chromatogram of scFv(7NP2). Most of the antibody was present in monomeric form and was eluted from the column at 11.5 mL.

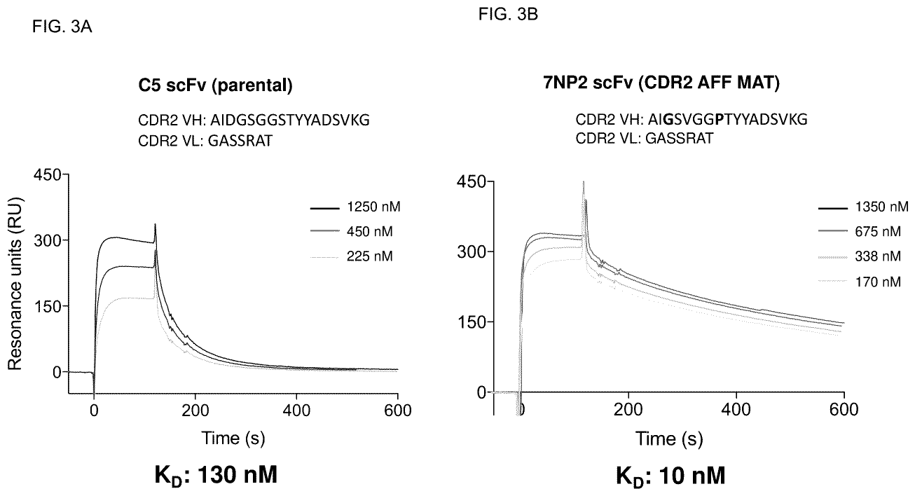

FIG. 3 shows the affinity of the anti-FAP scFvs C5 and 7NP2 to human FAP. FIG. 3A shows the results of BIAcore analysis using monomeric scFv(C5) on a chip coated with human FAP antigen at three different concentrations of the scFv: 1250 nM, 450 nM, and 225 nM. FIG. 3B shows the results of BIAcore analysis using monomeric scFv(7NP2) on a chip coated with human FAP antigen at four different concentrations of the scFv: 1350 nM, 675 nM, 338 nM, and 170 nM.

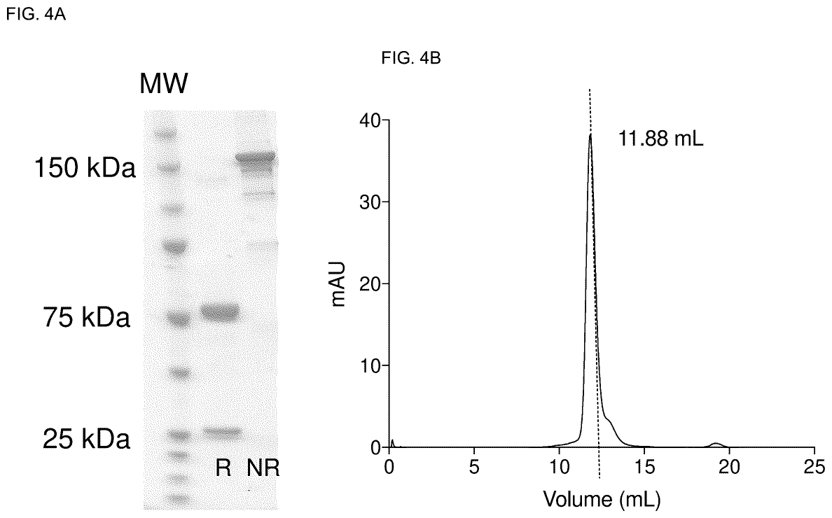

FIG. 4 shows the characterization of anti-FAP antibody 7NP2 in IgG1 format. FIG. 4A shows the results of SDS-PAGE analysis of 7NP2 IgG1. The IgG1 had the expected size of 150 kDa under non-reducing conditions and 25 and 75 kDa under reducing condition. FIG. 4B shows a size exclusion chromatogram of 7NP2 IgG1. The IgG1 was eluted from the column at 11.88 mL.

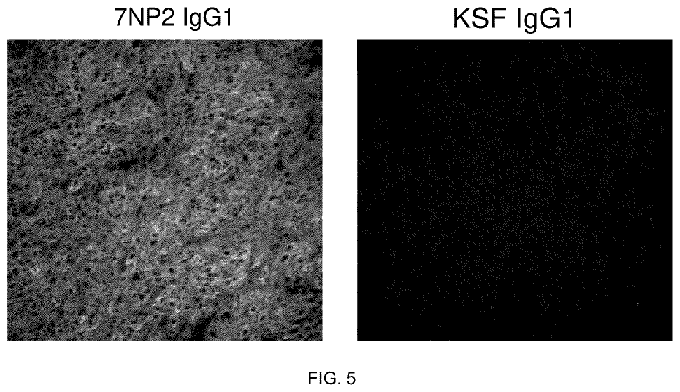

FIG. 5 shows the results of immunofluorescence staining of SKRC52-hFAP tissue slides using anti-FAP antibody 7NP2 IgG1-FITC and the anti-hen egg lysozyme antibody “KSF” (KSF IgG1-FITC; used as negative control). Anti-FAP antibody 7NP2 IgG1-FITC was capable of recognizing the human FAP on frozen tissue slides.

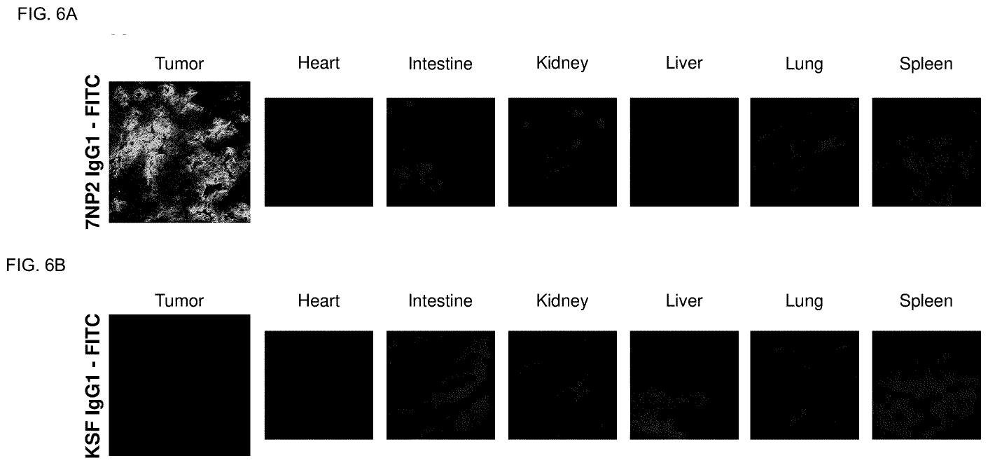

FIG. 6 shows the results of ex-vivo immunofluorescence staining of SKRC52-hFAP tumor bearing mouse organs with 7NP2 IgG1 (FIG. 6A). The negative control for the ex-vivo immunofluorescence analysis on SKRC52-hFAP tumor bearing mouse organs was antibody KSF in IgG1 format (FIG. 6B).

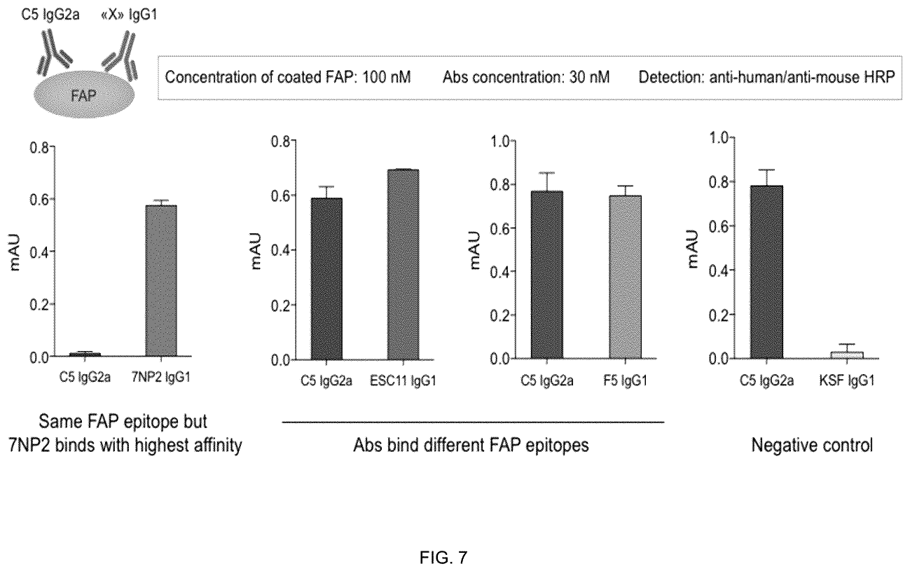

FIG. 7 shows the results of epitope analysis by ELISA. The C5 and 7NP2 antibody molecules shared the same epitope, with 7NP2 confirmed as having the higher affinity for human FAP, whereas the known anti-FAP antibodies F5 and ESC11 bound different epitopes on FAP. The KSF antibody in IgG1 format was used as negative control and showed no binding to human FAP.

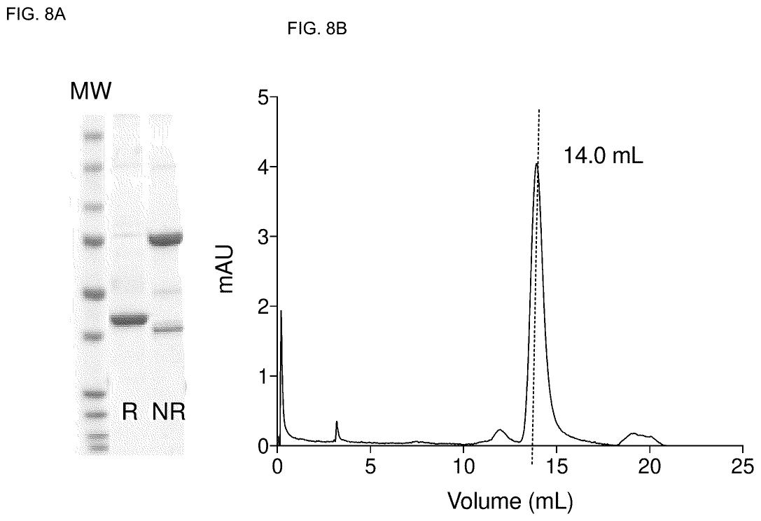

FIG. 8 shows the characterization of anti-FAP antibody 7NP2 in SIP format. FIG. 8A shows the results of SDS-PAGE analysis of 7NP2 SIP. The protein had the expected size of 77 kDa under non-reducing conditions and 38 kDa under reducing condition. FIG. 8B shows a size exclusion chromatogram for 7NP2 SIP. The 7NP2 SIP was eluted from the column at 14 mL.

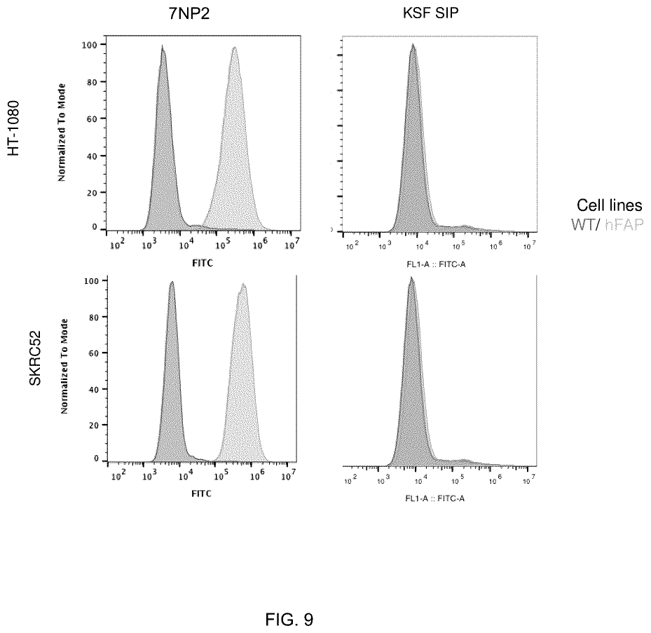

FIG. 9 shows the results of flow cytometry analysis evaluating binding of 7NP2 in SIP format to the wild-type HT-1080 and SKRC52 cell lines which do not express FAP and the same cell lines transduced to express FAP. Antibody 7NP2 in SIP format was capable of binding to the FAP-expressing cell lines with high specificity. Antibody KSF in SIP format was used as a negative control.

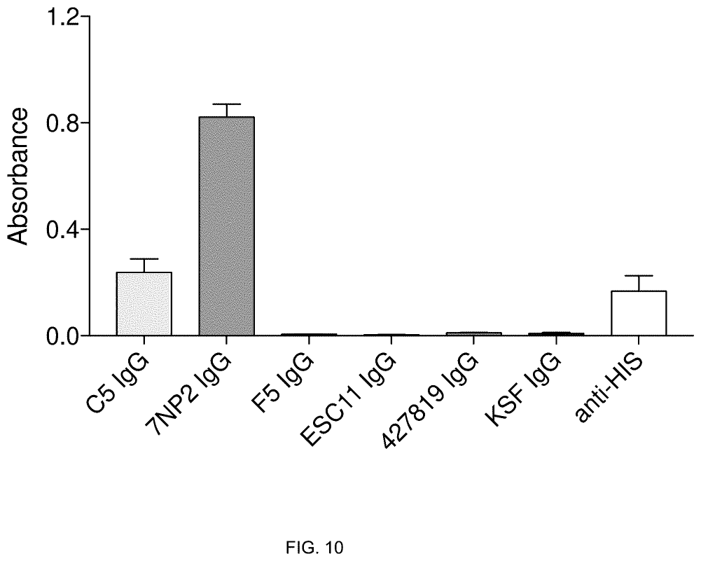

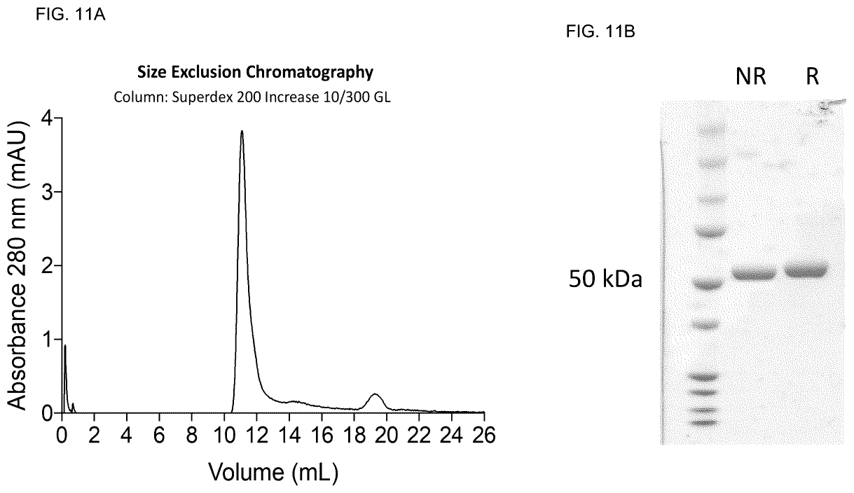

FIG. 10 shows the results of an ELISA evaluating binding of different anti-human FAP antibodies to canine FAP. Anti-FAP antibody molecules C5 (in IgG2a format), and antibodies 7NP2, F5, ESC11, and 427819 in IgG1 format were tested at a concentration of 5 μg/mL for binding to canine FAP. Canine FAP (caFAP) was coated onto the wells at a concentration 120 nM. Only the C5 and 7NP2 anti-FAP IgG antibodies showed binding to canine FAP (caFAP). The KSF antibody in IgG1 format was used as negative control, whereas an anti-His antibody was used as positive control for antigen coating

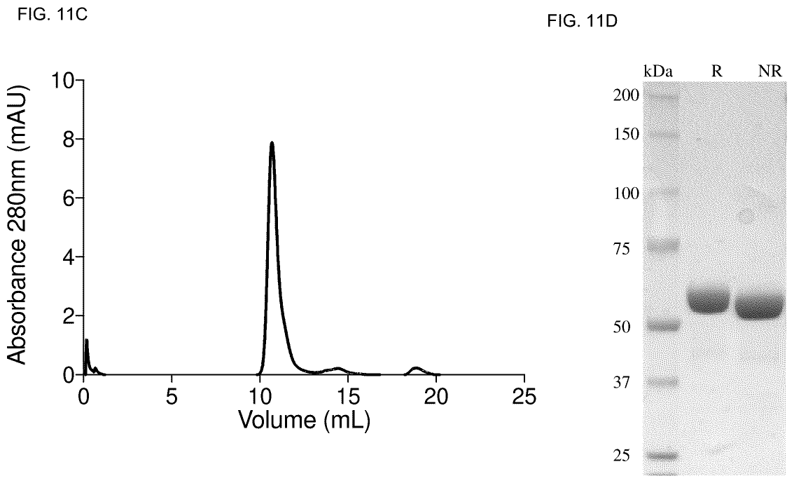

FIG. 11 shows the characterization of the IL2-7NP2-TNFmut conjugate. FIG. 11A shows a size exclusion chromatogram for the IL2-7NP2-TNFmut conjugate. The conjugate was eluted from the column at 11.10 mL. FIG. 11B shows the results of SDS-PAGE analysis of the IL2-7NP2-TNFmut conjugate. The conjugate had at the expected size of 60 kDa under non-reducing and reducing conditions, respectively. FIG. 11C shows the size exclusion chromatography profile of IL2-7NP2-TNF^{mut} v2. The conjugate was eluted from the column at 10.69 ml. FIG. 11D SDS-PAGE analysis of IL2-7NP2-TNF^{mut} v2 under non-reducing (NR) and reducing (R) conditions. The conjugate had at the expected size of 60 kDa under non-reducing and reducing conditions, respectively.

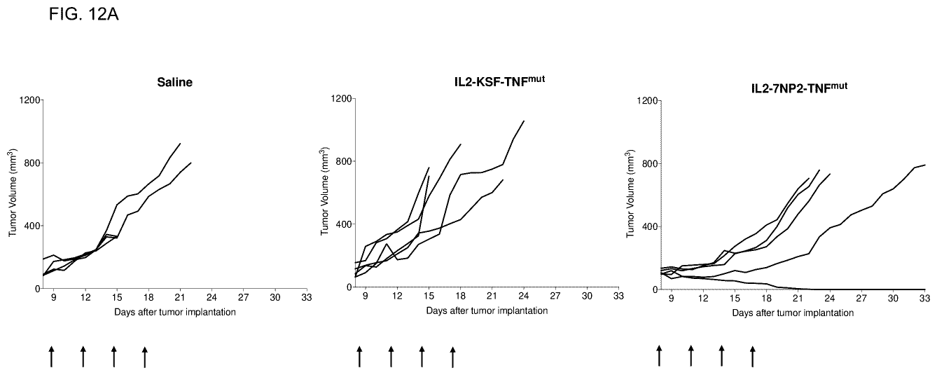

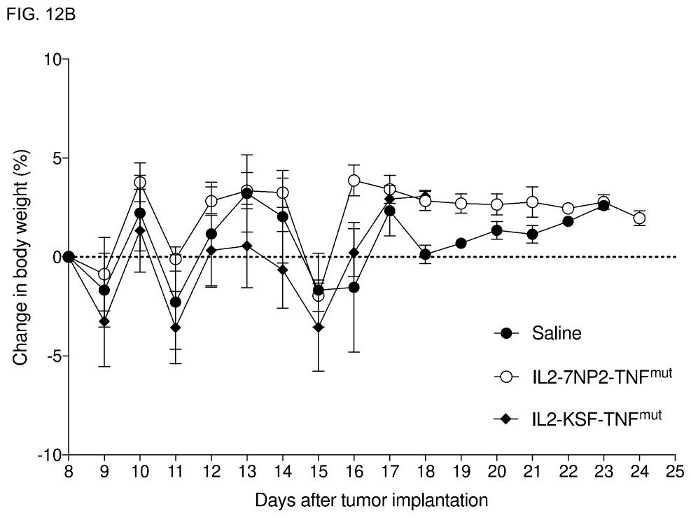

FIG. 12 shows (A) the results of therapy of SKRC52-hFAP tumors using the IL2-7NP2-TNFmut conjugate in a mouse tumor model and (B) the body weight changes of mice bearing SKRC52-hFAP tumor treated with saline, IL2-KSF-TNFmut and IL2-7NP2-TNFmut, respectively. Treatment with the IL2-7NP2-TNFmut conjugate resulted in tumor growth retardation and a ⅕ CR (complete response) compared with the negative controls (saline; IL2-KSF-TNFmut).

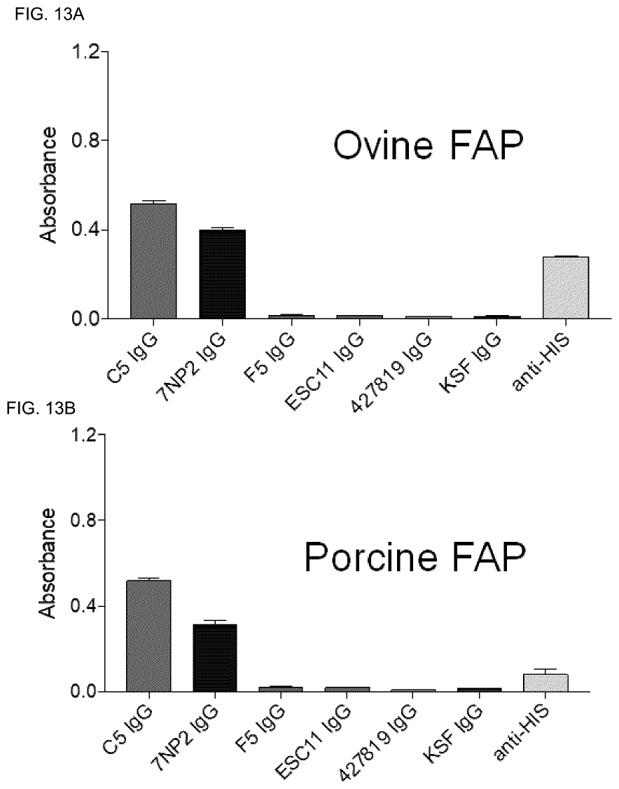

FIG. 13 shows the results of an ELISA evaluating binding of different anti-human FAP antibodies to ovine and porcine FAP. Anti-FAP antibody molecules C5 (in IgG2a format), and antibodies 7NP2, F5, ESC11, and 427819 in IgG1 format were tested at a concentration of 5 μg/mL for binding to ovine and porcine FAP. Ovine and porcine FAP were coated onto the wells at a concentration 120 nM. Only the C5 and 7NP2 anti-FAP IgG antibodies showed binding to ovine and porcine FAP. The KSF antibody in IgG1 format was used as negative control, whereas an anti-His antibody was used as positive control for antigen coating.

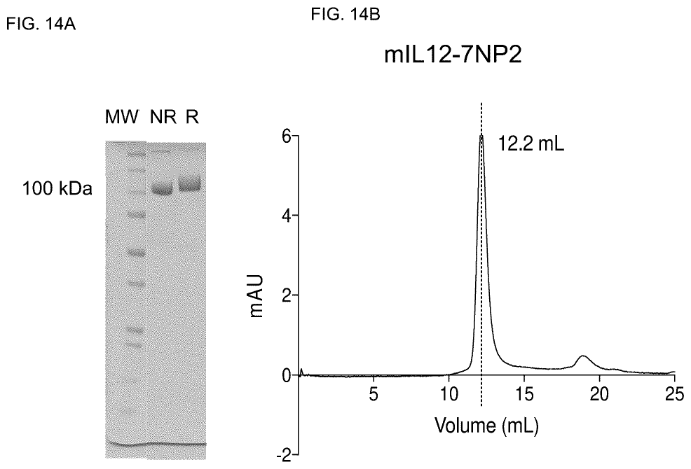

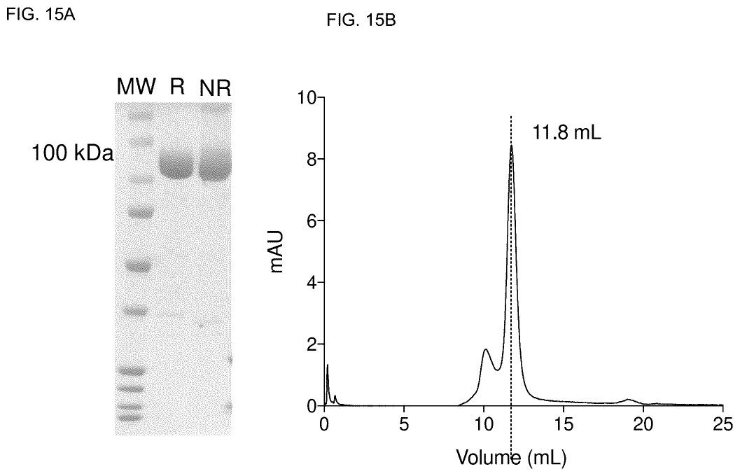

FIG. 14 shows the characterization of the miL12-7NP2 conjugate. FIG. 14A shows the results of SDS-PAGE analysis of the purified mIL12-7NP2 under non-reducing (NR) and reducing (R) conditions. FIG. 14B shows a size exclusion chromatogram for mIL12-7NP2. The mIL12-7NP2 was eluted from the column at 12.2 mL.

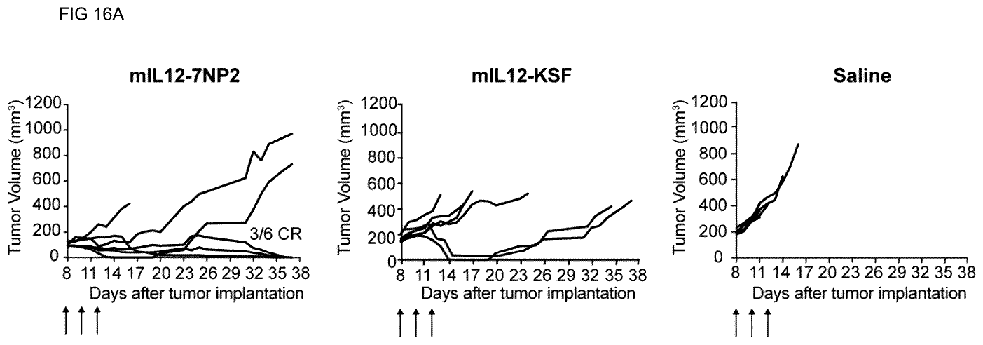

FIG. 15 shows the characterization of the hulL12-7NP2 conjugate. FIG. 15A shows the results of SDS-PAGE analysis of the purified hulL12-7NP2 conjugate under non-reducing (NR) and reducing (R) conditions. FIG. 15B shows a size exclusion chromatogram of hulL12-7NP2. The hulL12-7NP2 was eluted from the column at 11.8 mL.

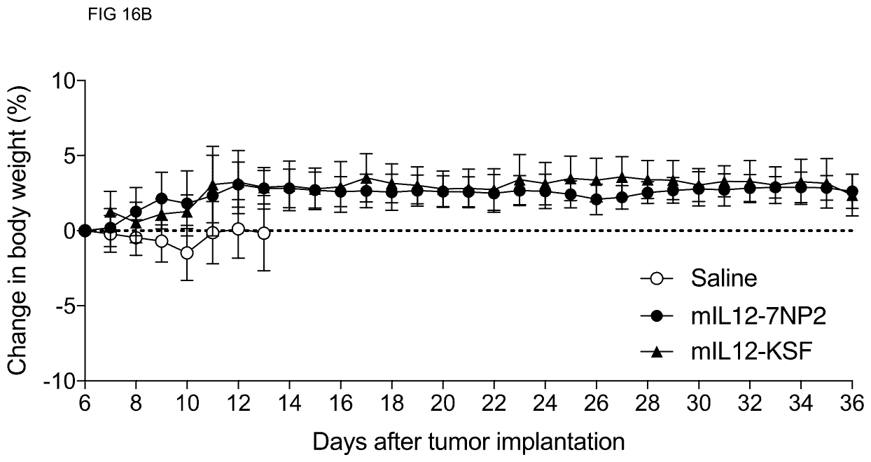

FIG. 16 shows the results of therapy of SKRC52-hFAP human renal cell carcinoma tumors using the mIL12-7NP2 conjugate in BALB/c nude mice. Data represent mean tumor volume±SEM, n=6 mice per group, CR=complete response. Treatment started when tumors reached a volume of 100 mm³, mice were injected three times intravenously every 48 hours with 8 μg of either mIL12-7NP2 (FIG. 16A), mIL12-KSF(FIG. 16B) or saline (FIG. 16C). FIG. 16D shows the body weight changes of mice treated with mIL12-7NP2, mIL12-KSF and saline respectively.

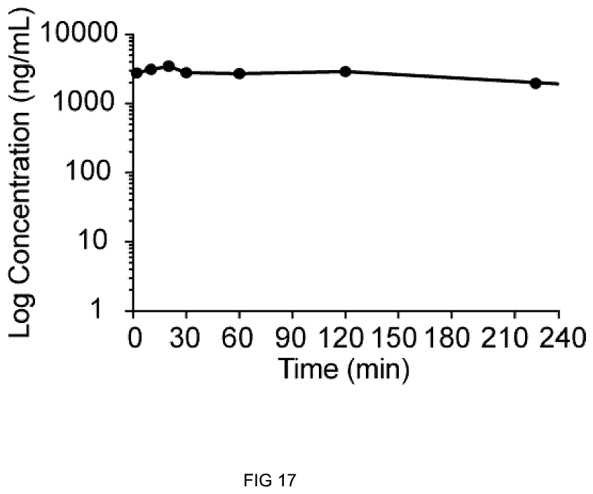

FIG. 17 shows the pharmacokinetics of 7NP2 in IgG1 format in Cynomolgus monkeys. Pharmacokinetics were evaluated in one cynomolgus monkey injected once at the dose of 0.1 mg/kg of 7NP2 IgG1. Blood samples were collected before dosing and at 2, 10, 20 and 30 min and 1, 2, and 4 h after treatment.

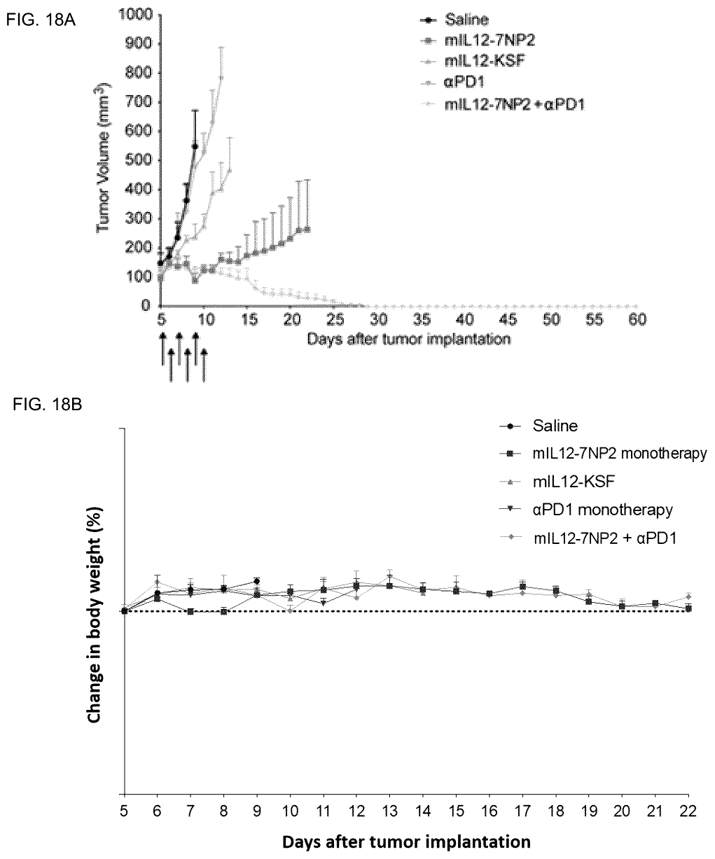

FIG. 18 shows the results of the therapy experiment in BALB/c mice bearing CT26-hFAP colon carcinoma tumors treated with mIL12-7NP2, mIL12-KSF, saline, aPD-1 or with mIL12-7NP2+αPD-1 combination regarding tumor volume (FIG. 18A) and body weight changes of mice in the same groups (FIG. 18B).

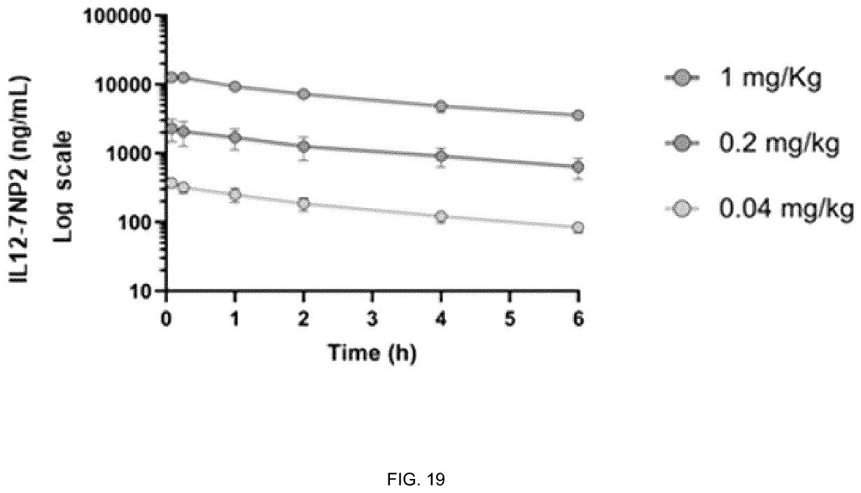

FIG. 19 shows the pharmacokinetics of IL12-7NP2 in Cynomolgus monkeys treated with three different dose levels: high dose group (1 mg/Kg), medium dose group (0.2 mg/Kg), low dose group (0.04 mg/Kg). Blood samples were collected before dosing and at 10 min and 1, 2, 4 and 6 h after treatment.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### Antibody Molecule

The present invention provides antibody molecules that bind human, ovine, porcine and canine FAP, preferably the extracellular domain of human, ovine, porcine and canine FAP. The extracellular domain of human, ovine, porcine and canine FAP may comprise or consist of the sequence set forth in SEQ ID NOs 1, 72, 73 and 2, respectively. The antibody molecule is preferably capable of binding to FAP expressed on the surface of a cell, such a cancer-associated fibroblast (CAF). Methods for determining binding an antigen, such as human, ovine, porcine or canine FAP, are known in the art and include ELISAs and flow cytometry, for example.

The antibody molecule preferably binds FAP specifically. The term “specific” may refer to the situation in which the antibody molecule will not show any significant binding to molecules other than its specific binding partner, here FAP. The term “specific” is also applicable where the antibody molecule is specific for particular epitopes, such as epitopes on FAP, that are carried by a number of antigens, in which case the antibody molecule will be able to bind to the various antigens carrying the epitope.

The antibody molecule, in scFv format, preferably binds human FAP with an affinity (K_{D}) of 10 nM, or with a higher affinity. The antibody molecule may further bind to canine, ovine and/or porcine FAP with the same affinity (K_{D}) as an anti-FAP antibody, in scFv format, consisting of the sequence set forth in SEQ ID NO: 11, or with an affinity that is higher. The binding affinity of an antibody molecule to a cognate antigen, such as human, canine, ovine or porcine FAP can be determined by surface plasmon resonance (SPR), such as Biacore, e.g. as detailed in the examples.

The antibody molecule is preferably monoclonal. The antibody molecule may be human or humanised, but preferably is a human antibody molecule.

The antibody molecule may be isolated, in the sense of being free from contaminants, such as antibodies able to bind other polypeptides, and/or serum components.

The antibody molecule may be natural or partly or wholly synthetically produced. For example, the antibody molecule may be a recombinant antibody molecule.

The antibody molecule may be an immunoglobulin, or an antigen-binding fragment thereof. For example, the antibody molecule may be an IgG, IgA, IgE or IgM molecule, preferably an IgG molecule, such as an IgG1, IgG2, IgG3 or IgG4 molecule, more preferably an IgG1 or IgG4 molecule, or an antigen-binding fragment thereof.

The antigen-binding site of an antibody molecule of the invention, such as an immunoglobulin or antigen-binding fragment thereof, binds FAP. The antigen-binding site may comprise three CDRs, such as the three light chain variable domain (VL) CDRs or three heavy chain variable domain (VH) CDRs, but preferably comprises six CDRs, three VL CDRs and three VH CDRs. The three VH domain CDRs of the antigen-binding site may be located within an immunoglobulin VH domain and the three VL domain CDRs may be located within an immunoglobulin VL domain. The antibody molecule may comprise one or two antigen-binding sites for FAP. Where the antibody molecule comprises two antigen-binding sites these are preferably identical. The antibody molecule thus may comprise one VH and one VL domain but preferably comprises two VH and two VL domains, i.e. two VH/VL domain pairs, as is the case in naturally-occurring immunoglobulin molecules, scFvs, diabodies and single-chain diabodies, for example.

The antigen-binding site of the antibody molecule preferably comprises the three VL domain CDRs and/or the three VH domain CDRs of antibody 7NP2. The VH and VL domain sequences of this antibody are set forth in SEQ ID NOs 9 and 10, respectively, and the sequences of the CDRs of the 7NP2 antibody may be readily determined from these VH and VL domain sequences by the skilled person using routine techniques.

The CDR sequences may, for example, be determined according to Kabat et al., “Sequences of Proteins of Immunological Interest,” National Institutes of Health, Bethesda, Md. (1991). In a preferred embodiment, the antigen-binding site of the antibody molecule comprises the HCDR1, HCDR2, and HCDR3 sequences set forth in SEQ ID NOs 3, 4 and 5, respectively, and the LCDR1, LCDR2 and LCDR3 sequences set forth in SEQ ID NOs 6, 7 and 8, respectively.

In a further preferred embodiment, the antigen-binding site may comprise the VH domain (SEQ ID NO: 9) and/or VL domain (SEQ ID NO: 10) of antibody 7NP2, but preferably comprises the VH domain and VL domain of antibody 7NP2.

The antibody molecule may also comprise a variant of a CDR, VH domain, VL domain, heavy chain or light chain sequence, as described herein. Suitable variants can be obtained by means of methods of sequence alteration, or mutation, and screening. In a preferred embodiment, an antibody molecule comprising one or more such variant sequences retain one or more of the functional characteristics of the parent antibody molecule, such as binding specificity and/or binding affinity for human, ovine, porcine and/or canine FAP, preferably human, ovine and porcine and canine FAP. For example, an antibody molecule comprising one or more variant sequences preferably binds to human, ovine and porcine and/or canine FAP with the same affinity as, or a higher affinity than, the (parent) antibody molecule. The parent antibody molecule is antibody molecule which does not comprise the amino acid substitution(s), deletion(s), and/or insertion(s) which has (have) been incorporated into the variant antibody molecule.

The antibody molecule may comprise a VH domain which has at least 70%, more preferably at least 75%, at least 80%, at least 85%, at least 90%, at least 95%, at least 96%, at least 97%, at least 98%, at least 99% or 100%, sequence identity to the VH domain of antibody 7NP2 (SEQ ID NO: 9).

The antibody molecule may comprise a VL domain with at least 70%, more preferably one of at least 75%, at least 80%, at least 85%, at least 90%, at least 95%, at least 96%, at least 97%, at least 98%, at least 99% or 100%, sequence identity to the VL domain of antibody 7NP2 (SEQ ID NO: 10).

The antibody molecule may comprise a heavy chain which has at least 70%, more preferably at least 75%, at least 80%, at least 85%, at least 90%, at least 95%, at least 96%, at least 97%, at least 98%, at least 99% or 100%, sequence identity to the heavy chain of antibody 7NP2 in IgG1 format (SEQ ID NO: 13).

The antibody molecule may comprise a heavy chain which has at least 70%, more preferably at least 75%, at least 80%, at least 85%, at least 90%, at least 95%, at least 96%, at least 97%, at least 98%, at least 99% or 100%, sequence identity to the heavy chain of antibody 7NP2 in IgG4 format (SEQ ID NO: 74).

The antibody molecule may comprise a light chain which has at least 70%, more preferably at least 75%, at least 80%, at least 85%, at least 90%, at least 95%, at least 96%, at least 97%, at least 98%, at least 99% or 100%, sequence identity to the light chain of antibody 7NP2 (SEQ ID NO: 14). Sequence identity is commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm (Nucl. Acids Res. (1997) 25 3389-3402) may be used.

Variants of the CDRs, VH domain, VL domain, heavy chain or light chain sequence disclosed herein comprising one or more, e.g. less than 20 alterations, less than 15 alterations, less than 10 alterations or less than 5 alterations, 4, 3, 2 or 1, amino acid alterations (addition, deletion, substitution and/or insertion of an amino acid residue) may also be employed in antibody molecules according to the invention. Suitable variants can be obtained by means of methods of sequence alteration, or mutation, and screening.

Alterations may be made in one or more framework regions and/or one or more CDRs. In particular, alterations may be made in HCDR1, HCDR2 and/or HCDR3, or in one or more framework regions of the heavy or light chain of the antibody molecule.

In one example, the heavy chain of an antibody molecule of the invention may comprise a C-terminal lysine residue as shown e.g. in SEQ ID NOs 13 and 74, or said lysine residue may be deleted.

As noted above, the antibody molecule may be a whole antibody or a fragment thereof, in particular an antigen-binding fragment thereof.

Antigen-binding fragments of immunglobulins include (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward et al. (1989) Nature 341, 544-546; McCafferty et al., (1990) Nature, 348, 552-554; Holt et al. (2003) Trends in Biotechnology 21, 484-490), which consists of a VH or a VL domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al. (1988) Science, 242, 423-426; Huston et al. (1988) PNAS USA, 85, 5879-5883); (viii) bispecific single chain Fv dimers (WO1993/011161), (ix) “diabodies”, multivalent or multispecific fragments constructed by gene fusion (WO2013/014149; WO94/13804; Holliger et al. (1993a), Proc. Natl. Acad. Sci. USA 90 6444-6448) and (x) “single-chain diabodies” wherein two sets of VH and VL domains are connected together in sequence on the same polypeptide chain (Konterman & Muller, 1999). Fv, scFv or diabody molecules may be stabilized by the incorporation of disulphide bridges linking the VH and VL domains (Reiter et al. (1996), Nature Biotech, 14, 1239-1245). Minibodies comprising a scFv joined to a CH3 domain may also be made (Hu et al. (1996), Cancer Res., 56(13):3055-61). Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group.

A single chain Fv (scFv) may be comprised within a mini-immunoglobulin or small immunoprotein (SIP), e.g. as described in (Li et al., (1997), Protein Engineering, 10: 731-736). A SIP may comprise an scFv molecule fused to the CH4 domain of the human IgE secretory isoform IgE-S2 (ε_{S2}-CH4; Batista et al., (1996), J. Exp. Med., 184: 2197-205) forming a homo-dimeric mini-immunoglobulin antibody molecule.

Preferably, the antibody molecule comprises or consists of a single-chain Fv (scFv), a small immunoprotein, a diabody, a single-chain diabody or a (whole) IgG molecule, such as an IgG1 or IgG4 molecule.

Where the antibody molecule is an scFv, the VH and VL domains of the antibody are preferably linked by a 14 to 20 amino acid linker. For example, the VH and VL domains may be linked by an amino acid linker which is 14, 15, 16, 17, 18, 19, or 20 amino acid in length. Suitable linker sequences are known in the art and include the linker sequence set forth in SEQ ID NOs: 12 and 83.

In a preferred embodiment, the antibody molecule of the invention in scFv format comprises or consists of the sequence set forth in SEQ ID NO: 11.

Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen-binding site: antigen-binding sites are formed by the association of the first domain of one polypeptide within the multimer with the second domain of another polypeptide within the multimer (WO94/13804; Holliger and Winter, 1997; Holliger et al., 1993).

In a diabody or single-chain diabody, a heavy chain variable domain (VH) is connected to a light chain variable domain (VL) on the same polypeptide chain. The VH and VL domains are connected by a peptide linker that is too short to allow pairing between the two domains. This forces pairing with the complementary VH and VL domains of another chain.

Where the antibody molecule is a diabody or single-chain diabody, the VH and VL domains are preferably linked by a 5 to 12 amino acid linker. For example, the VH and VL domains may be linked by an amino acid linker which is 5, 6, 7, 8, 9, 10, 11, or 12 amino acids in length. Preferably, the amino acid linker is 5 amino acids in length. Suitable linker sequences are known in the art and include the linker sequence set forth in SEQ ID NO: 40.

In a preferred embodiment, the antibody molecule of the invention in diabody format has the sequence set forth in SEQ ID NO: 38.

In a single-chain diabody, two sets of VH and VL domains are connected together in sequence on the same polypeptide chain. For example, the two sets of VH and VL domains may be assembled in a single-chain sequence as follows: (VH-VL)—(VH-VL), where the brackets indicate a set. The two sets of VH and VL domains are connected as a single-chain by a long or ‘flexible’ peptide linker. This type of peptide linker sequence is long enough to allow pairing of the VH and VL domains of the first set with the complementary VH and VL domains of the second set. Generally, a long or ‘flexible’ linker is 15 to 20 amino acids. Suitable flexible linker sequences are known in the art and include the linker sequence set forth in SEQ ID NO: 69.

In a preferred embodiment, the antibody molecule of the invention in single-chain diabody format has the sequence set forth in SEQ ID NO: 39.

Where the antibody is a small immunoprotein (SIP) e.g. as described in (Li et al., (1997), Protein Engineering, 10: 731-736), the VL domain of the scFv antibody is preferably linked to the CH4 domain of human IgE (Batista et al., (1996), J. Exp. Med., 184: 2197-205) via a 2 to 20 amino acid linker, more preferably a 2 to 10 amino acid linker. Suitable linker sequences are known in the art and include the linker sequence set forth in SEQ ID NO: 16.

In a further preferred embodiment, the antibody molecule of the invention in SIP format has the sequence set forth in SEQ ID NO: 15.

### Conjugate

Conjugates of the invention comprise an antibody molecule of the invention and a therapeutic or diagnostic agent. The therapeutic agent may be an anti-inflammatory agent, a pro-inflammatory agent, a biocidal molecule, a cytotoxic molecule, a radioisotope, a photosensitizer, an enzyme, a hormone, or an immunosuppressive agent. Preferably, the therapeutic agent is a biocidal molecule, a cytotoxic molecule, a radioisotope, an anti-inflammatory agent, a pro-inflammatory agent, or an immunosuppressive agent. The biocidal molecule, cytotoxic molecule, anti-inflammatory agent, a pro-inflammatory agent, or immunosuppressive agent may be a cytokine. In particular, the therapeutic agent conjugated to the antibody molecule may have both immunosuppressive and anti-inflammatory activity. Most preferably, the therapeutic agent conjugated to the antibody molecule of the invention is a pro-inflammatory or anti-inflammatory agent, in particular a pro-inflammatory or anti-inflammatory cytokine.

Pro-inflammatory cytokines which may be conjugated to an antibody molecule of the invention include interleukin-2 (IL2), interleukin-12 (IL12), interleukin-15 (IL15), interferon (IFN), such as IFNγ, and tumour necrosis factor (TNF), such as TNFα, as well as mutants or variants thereof. The sequence of IL2 is set forth in SEQ ID NO: 34. The sequence of single-chain IL12 as disclosed in WO2013/014149 is set forth in SEQ ID NO: 70. The sequence of a TNFα mutant which may be conjugated to an antibody molecule of the invention is set forth in SEQ ID NO: 35. The sequence of a IFN gamma mutant which may be conjugated to an antibody molecule of the invention is set forth in SEQ ID NO: 71. The sequences of the Sushi Domain (SD) of the IL15 Receptor alpha and of IL15 which may be conjugated to an antibody molecule of the invention are set forth in SEQ ID NOs: 77 and 78, respectively. The sequences of the remaining cytokines, as well as variants thereof which may be employed in the present invention, are known in the art.

Anti-inflammatory cytokines which may be conjugated to an antibody molecule of the invention include IL10, IL4, IL22 and mutants or variants thereof. The sequences of these cytokines, as well as variants thereof which may be employed in the present invention, are also known in the art.

A therapeutic agent may be conjugated to the N-terminus or C-terminus of the antibody molecule or both. Where a therapeutic agent is conjugated to both the N-terminus and the C-terminus of the antibody molecule, the therapeutic agents may be the same or different but preferably are different. Where the therapeutic agent is conjugated to the N-terminus of the antibody molecule, the C-terminus may be “free”, i.e. not conjugated to another moiety. Similarly, where the therapeutic agent is conjugated to the C-terminus of the antibody molecule, the N-terminus may be “free”, i.e. not conjugated to another moiety.

In a preferred embodiment, the antibody molecule, preferably in single-chain diabody format, is conjugated to interleukin 12 (IL12). In a preferred embodiment, the antibody molecule is conjugated at its N-terminus to IL12. In more preferred embodiment, the conjugate comprises or consists of the sequence set forth in SEQ ID NO: 41.

In another preferred embodiment, the antibody molecule, preferably in IgG4 format, is conjugated to a mutant of interferon gamma (INFγ Mut), preferably at the C-terminus of the heavy chain of the antibody molecule. In a more preferred embodiment, the conjugate comprises or consists of the light and heavy chain sequences set forth in SEQ ID NOs 14 and/or 37.

In a yet further preferred embodiment, the antibody molecule, preferably in scFv format, is conjugated, preferably at its N-terminus, to interleukin 2 (IL2) and, preferably at its C-terminus, to a mutant of tumour necrosis factor alpha (TNFα). In a more preferred embodiment, the conjugate comprises or consists of the sequence set forth in SEQ ID NO: 31 or 82.

In another preferred embodiment, the antibody molecule, preferably in diabody format, is conjugated to interleukin 2 (IL2). In a preferred embodiment, the antibody molecule is conjugated at its C-terminus to IL2. In a more preferred embodiment, the conjugate comprises or consists of the sequence set forth in SEQ ID NO 76.

In another preferred embodiment, the antibody molecule, preferably in single-chain diabody format, is conjugated to the sushi domain of the IL15 Receptor alpha (SD) and interleukin 15 (I15). In a preferred embodiment, the antibody molecule is conjugated at its C-terminus to the sushi domain of the IL15 Receptor alpha (SD) and the IL15 is conjugated to the C-terminus of the sushi domain. In a more preferred embodiment, the conjugate comprises or consists of the sequence set forth in SEQ ID NO: 79.

A diagnostic agent conjugated to the antibody molecule of the invention may be a detectable label, such as a radioisotope, e.g. a non-therapeutic radioisotope.

Radioisotopes which may be conjugated to an antibody molecule of the invention include isotopes such as ^{94m}Tc, ^{99m}Tc, ¹⁸⁶Re, ¹⁸⁸Re, ²⁰³Pb, ⁶⁷Ga, ⁶⁸Ga, ⁴⁷Sc, ¹¹¹In, ⁹⁷Ru, ⁶²Cu, ⁶⁴Cu, ⁸⁶Y, ⁸⁸Y, ⁹⁰Y, ¹²¹Sn, ¹⁶¹Tb, ¹⁵³Sm, ¹⁶⁶Ho, ¹⁰⁵Rh, ¹⁷⁷Lu, ¹²³I, ¹²⁴I, ¹²⁵I, ¹³¹I, ¹⁸F, ²¹¹At and ²²⁵Ac. Preferably, positron emitters, such as ¹⁸F and ¹²⁴I, or gamma emitters, such as ^{99m}Tc, ¹¹¹1n and ¹²³I, are used for diagnostic applications (e.g. for PET), while beta-emitters, such as ¹³¹I, ⁹⁰Y and ¹⁷⁷Lu, are preferably used for therapeutic applications. Alpha-emitters, such as ²¹¹At and ²²⁵Ac may also be used for therapy. In one example, the antibody molecule may be conjugated to ¹⁷⁷Lu, ¹³¹I, or ⁹⁰Y.

The antibody molecule may be conjugated with the therapeutic agent by means of a peptide bond or linker as described herein. Other means for conjugation include chemical conjugation, especially cross-linking using a bifunctional reagent (e.g. employing DOUBLE-REAGENTS™ Cross-linking Reagents Selection Guide, Pierce).

### Linkers

The antibody molecule, e.g. scFv or IgG, and the therapeutic or diagnostic agent or molecule may be connected to each other directly, for example through any suitable chemical bond, but preferably are connected via a peptide linker. The chemical bond may be, for example, a covalent or ionic bond. Examples of covalent bonds include peptide bonds (amide bonds) and disulphide bonds.

Where the therapeutic or diagnostic agent is connected to the antibody molecule via a peptide linker, the peptide linker may be a short (2-30, preferably 10-20) residue stretch of amino acids. Suitable examples of peptide linker sequences are known in the art. One or more different linkers may be used. Exemplary linkers are set forth in SEQ ID NOs 32, 33, 80 and 83, for example. In one embodiment, the linker may be a cleavable linker.

Where the antibody molecule and therapeutic or diagnostic agent are connected via a peptide bond or peptide linker, the conjugate may be produced (secreted) as a single chain polypeptide, such as a fusion protein.

### Methods of Treatment

As explained above, the presence of FAP and FAP-expressing fibroblasts has been shown to be associated with a number of diseases and disorders, including diseases characterised by inflammation and/or angiogenesis, such as cancer, as well as inflammatory disorders and autoimmune diseases.

An antibody molecule or conjugate of the invention may therefore be for use as a medicament.

In particular, the antibody molecule or conjugate may be for use in a method of treatment (which may include prophylactic treatment) of the human or animal body (e.g. a domestic dog).

Also provided is a method of treating a disease or disorder in a patient, wherein the method comprises administering to the patient a therapeutically effective amount of the antibody molecule or conjugate.

Further provided is the use of the antibody molecule or conjugate in the manufacture of a medicament for use in the treatment of a disease or disorder in a patient.

The patient may be a human patient or may be an animal patient, preferably a domestic dog.

Treatment may be any treatment or therapy in which some desired therapeutic effect is achieved, for example, the inhibition or delay of the progress of the disease or disorder, and includes a reduction in the rate of progress, a halt in the rate of progress, amelioration of the disease or disorder, cure or remission (whether partial or total) of the disease or disorder, preventing, ameliorating, delaying, abating or arresting one or more symptoms and/or signs of the disease or disorder or prolonging survival of an individual or patient beyond that expected in the absence of treatment.

Treatment as a prophylactic measure (i.e. prophylaxis) is also included. For example, an individual susceptible to or at risk of the occurrence or re-occurrence of a disease or disorder may be treated as described herein. Such treatment may prevent or delay the occurrence or re-occurrence of the disease or disorder in the individual.

A method of treatment as described may comprise administering at least one further treatment to the individual in addition to the antibody molecule or conjugate. The antibody molecule or conjugate may thus be administered to an individual alone or in combination with one or more other treatments for the disease or disorder in question. Where the antibody molecule or conjugate is administered to the individual in combination with another treatment, the additional treatment may be administered to the individual concurrently with, sequentially to, or separately from the administration of the antibody molecule or conjugate. Where the additional treatment is administered concurrently with the antibody molecule or conjugate, the antibody molecule or conjugate and additional treatment may be administered to the patient as a combined preparation. For example, the additional therapy may be a known therapy or therapeutic agent for the disease or disorder to be treated.

In a preferred embodiment, the conjugate of the invention is administered to a patient in combination with a JAK inhibitor.

The present invention thus provides a conjugate of the invention for use in a method of treating an inflammatory disorder, autoimmune disease, or cancer in a patient, wherein the method further comprises administering a JAK inhibitor to the patient. Also provided is a method of treating an inflammatory disorder, autoimmune disease, or cancer in a patient, wherein the method comprises administering a conjugate of the invention and a JAK inhibitor to the patient. The JAK inhibitor may be administered to the patient concurrently with, sequentially to, or separately from the administration of the conjugate. Further provided is the use of a conjugate of the invention for the manufacture of a medicament for the treatment of an inflammatory disorder, inhibiting angiogenesis, treating cancer, and/or treating an autoimmune disease, wherein treatment comprises administering the conjugate and a JAK inhibitor to the patient.

The JAK inhibitor is preferably selected from the group consisting of: ruxolitinib, baricitinib, tofacitinib, fedratinib, momelotinib, pacritinib, fligotinib, upadacitinib, itacitinib, decernotinib, peficitinib, deucravacitinib, abrocitinib, NDI-031301 and ritlecitinib. Most preferably, the JAK inhibitor is ruxolitinib.

In a further preferred embodiment, the conjugate of the invention is administered to a patient in combination with an immunomodulatory agent, such as an anti-PD-1 antibody, anti-PD-L1 antibody, anti-LAG-3 antibody, anti-TIGIT antibody, or anti-TIM-3 antibody. Most preferably, the immunomodulatory agent is an anti-PD-1 antibody.

The present invention thus provides a conjugate of the invention for use in a method of treating cancer in a patient, wherein the method further comprises administering an immunomodulatory agent to the patient. Also provided is a method of treating a cancer in a patient, wherein the method comprises administering a conjugate of the invention and an immunomodulatory agent to the patient. The immunomodulatory agent may be administered to the patient concurrently with, sequentially to, or separately from the administration of the conjugate. Further provided is the use of a conjugate of the invention for the manufacture of a medicament for the treatment of cancer, wherein treatment comprises administering the conjugate and an immunomodulatory agent to the patient.

Anti-PD-1, anti-PD-L1, anti-LAG-3, anti-TIGIT, and anti-TIM-3 antibodies are known in the art and are available to the skilled person. A number of anti-PD-1 and anti-PDL-1 antibodies are licensed for the treatment of cancer in human patients and can be employed in treatment of cancer in a patient in combination with a conjugate of the invention.

The conjugate administered to the patient in combination with a JAK inhibitor or immunomodulatory agent preferably comprises a single-chain diabody conjugated at its N-terminus to IL12, wherein the conjugate comprises the CDRs and/or VH and VL domains of the 7NP2 anti-FAP antibody, as disclosed herein. Most preferably the conjugate comprises, or consists of, the sequence set forth in SEQ ID NO: 41. Also provided is an antibody molecule of the invention for delivering an agent conjugated to the antibody molecule to the site of a disease or disorder in a patient. Similarly provided is a method of delivering an agent conjugated to the antibody molecule to the site of a disease or disorder in a patient, wherein the method comprises administering the antibody molecule to the patient.

The disease or disorder to be treated may be a disease or disorder characterised by angiogenesis, such as cancer, an autoimmune disease or inflammatory disorder.

The disease or disorder to be treated using an antibody molecule or conjugate of the invention may be any disease or disorder characterised by, or associated with, the expression of FAP. As explained above, expression of FAP is very limited in adult human tissues and is therefore expected to represent a disease-specific target for therapy in diseases or disorders characterised by expression of FAP. For example, the disease or disorder may be characterised by, or associated with, the presence of FAP-expressing fibroblasts, as is known to be the case for a wide variety of cancers. In addition, or alternatively, the disease to be treated may be cancer, wherein the cancer cells express FAP. As a further alternative, the disease or disorder may be associated with, or characterised by, the presence of fibrotic tissue comprising expression of FAP, e.g. in the extracellular matrix, for example as a result of the presence of FAP-expressing fibroblasts.

Inflammatory disorders include any disease or disorder which is characterised by an inflammatory abnormality. Such diseases include, for example, immune system disorders, such as autoimmune diseases, and cancer.

The disease to be treated using an antibody molecule or conjugate of the invention may be cancer, as well as other tumours and neoplastic conditions.

Exemplary cancers include any type of solid or non-solid cancer or malignant lymphoma and especially liver cancer, lymphoma, leukaemia (e.g. acute myeloid leukaemia), sarcomas, skin cancer, bladder cancer, breast cancer, uterine cancer, ovarian cancer, prostate cancer, lung cancer, colorectal cancer, cervical cancer, head and neck cancer, oesophageal cancer, pancreatic cancer, renal cancer, stomach cancer and cerebral cancer. Cancers may be familial or sporadic. Cancers may be metastatic or non-metastatic. The cancer, tumour, or neoplastic condition may express FAP or comprise FAP-expressing fibroblasts.

Autoimmune diseases which may be treated using an antibody molecule or conjugate of the invention herein include lupus erytematosus, rheumatoid arthritis, and psoriatic arthritis.

An inflammatory or autoimmune disease which may treated using an antibody molecule or conjugate of the invention is inflammatory bowel disease (IBD), such Crohn's disease or ulcerative colitis. A further disease which is known to be associated with FAP expression and thus may be treated using an antibody molecule or conjugate of the invention is osteoarthritis.

### Methods of Detection or Diagnosis

The antibody molecules and conjugates are expected to be suitable for detecting FAP in vivo and in vitro, and thus find application in the imaging, detection and diagnosis of disease characterised by, or associated with, expression of FAP, e.g. as the result of the presence of FAP-expressing fibroblasts.

The present invention therefore also relates to the use of an antibody molecule or conjugate of the invention for detecting FAP, e.g. cells, such as fibroblasts, expressing FAP on their cell surface, either in vitro or in vivo. The conjugate preferably comprises a detectable label to aid detection. The preparation of suitable conjugates is described elsewhere herein. Alternatively, binding of the antibody molecule to FAP may be detected using a secondary antibody or other detection reagent. Where the antibody molecule is conjugated to a radioisotope, binding of the antibody molecule to FAP in the patient may be detected using scintigraphy.

Also provided is an in vitro method for detecting FAP, the method comprising incubating the antibody molecule or conjugate with a sample obtained from an individual, e.g. a human patient, and detecting binding of the antibody molecule or conjugate to the sample, e.g. cells (such as fibroblasts) present in the sample, wherein binding of the antibody molecule or conjugate to the sample indicates the presence of FAP. Methods for determining binding of an antibody molecule or antigen to a sample are known in the art and include, for example, ELISAs, flow cytometry, and immunostaining of tissue samples.

Further provided is the antibody molecule or conjugate for use in a method of detecting FAP in vivo, the method comprising administering the antibody molecule or conjugate to an individual, e.g. a human patient, wherein localisation of the antibody molecule or conjugate at a site in the individual, indicates expression of FAP at said site.

As FAP is rarely expressed in adult human tissues but is known to be expressed on disease-associated fibroblast, such as cancer-associated fibroblast, and on tumour cells, for example, the antibody molecules and conjugates of the invention are also expected to find application in the detection of diseases and disorders characterised by expression of FAP. Thus, the present invention also provides an antibody molecule or conjugate of the invention for use a detection agent, diagnostic, or imaging agent.

Thus, the present invention also provides the antibody molecule or conjugate for use in a method of imaging, detecting, or diagnosing a disease or disorder in a patient.

Also provided is a method of imaging, detecting, or diagnosing a disease or disorder in a patient comprising administering an antibody molecule or conjugate of the invention to the patient.

Further provided is the use of an antibody molecule or conjugate of the invention in the manufacture of a diagnostic product for use in the detection or diagnosis of a disease or disorder.

The disease or disorder is preferably characterised by expression of FAP, such as the presence of FAP-expressing fibroblasts, and may be a disease or disorder as described herein, such as an inflammatory disorder, angiogenesis, cancer, and/or an autoimmune disease Pharmaceutical compositions Whilst an antibody molecule or conjugate may be administered alone, antibody molecules and conjugates will typically be administered in the form of a pharmaceutical composition. Thus, a further aspect of the present invention relates to a pharmaceutical composition comprising at least one antibody molecule or conjugate of the invention and at least one other component, such as a pharmaceutically acceptable excipient. A method comprising formulating an antibody molecule or conjugate into a pharmaceutical composition is also provided.

Pharmaceutical compositions may comprise, in addition to the antibody molecule or conjugate, a pharmaceutically acceptable excipient, carrier, buffer, stabilizer or other materials well known to those skilled in the art. The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. The precise nature of the carrier or other material will depend on the route of administration, which may be by infusion, injection or any other suitable route, as discussed below.

For parenteral, for example subcutaneous or intravenous administration, e.g. by injection, the pharmaceutical composition comprising the antibody molecule or conjugate may be in the form of a parenterally acceptable aqueous solution which is pyrogen-free and has suitable pH, isotonicity and stability. Those of relevant skill in the art are well able to prepare suitable solutions using, for example, isotonic vehicles, such as Sodium Chloride Injection, Ringer's Injection, Lactated Ringer's Injection. Preservatives, stabilizers, buffers, antioxidants and/or other additives may be employed as required, including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagine, histidine, arginine, or lysine; monosaccharides, disaccharides and other carbohydrates including glucose, mannose or dextrins; chelating agents, such as EDTA; sugars, such as sucrose, mannitol, trehalose or sorbitol; salt-forming counter-ions, such as sodium; metal complexes (e.g. Zn-protein complexes); and/or non-ionic surfactants, such as TWEEN™, PLURONICS™ or polyethylene glycol (PEG).

In some embodiments, the antibody molecules or conjugates may be provided in a lyophilised form for reconstitution prior to administration. For example, lyophilised antibody molecules or conjugates may be re-constituted in sterile water and mixed with saline prior to administration to an individual.

Administration may be in a “therapeutically effective amount”, this being sufficient to show benefit to an individual. The actual amount administered, and rate and time-course of administration, will depend on the nature and severity of the disease or disorder being treated, the particular individual being treated, the clinical condition of the individual, the cause of the disorder, the site of delivery of the composition, the type of antibody molecule or conjugate, the method of administration, the scheduling of administration and other factors known to medical practitioners. Prescription of treatment, e.g. decisions on dosage etc., is within the responsibility of general practitioners and other medical doctors. Appropriate doses of antibody molecules are well known in the art (Ledermann et al., 1991; Bagshawe et al., 1991). Specific dosages indicated herein, or in the Physician's Desk Reference (2003) as appropriate for an antibody molecule being administered, may be used. Appropriate doses for conjugates are also known or can be determined. For example, a therapeutically effective amount or suitable dose of an antibody molecule or conjugate can be determined by comparing in vitro activity and in vivo activity in an animal model, such as a domestic dog, a pig, or a sheep. Methods for extrapolation of effective dosages in domestic dogs, pigs and sheep, as well as other test animals to humans are known. The precise dose will depend upon a number of factors, including whether the size and location of the area to be treated, and the precise nature of the antibody molecule or conjugate.

Treatments may be repeated at daily, twice-weekly, weekly or monthly intervals, at the discretion of the physician. The treatment schedule for an individual may be dependent on the pharmacokinetic and pharmacodynamic properties of the antibody molecule or conjugate, the route of administration and the nature of the condition being treated.

Treatment may be periodic, and the period between administrations may be about two weeks or more, e.g. about three weeks or more, about four weeks or more, about once a month or more, about five weeks or more, or about six weeks or more. For example, treatment may be every two to four weeks or every four to eight weeks. Suitable formulations and routes of administration are described above.

A pharmaceutical composition may be administered alone or in combination with other treatments, either simultaneously or sequentially dependent upon the condition to be treated.

### Kits

Another aspect of the invention provides a therapeutic kit for use in the treatment of a disease or disorder comprising an antibody molecule or conjugate as described herein. The components of a kit are preferably sterile and in sealed vials or other containers.

A kit may further comprise instructions for use of the components in a method described herein. The components of the kit may be comprised or packaged in a container, for example a bag, box, jar, tin or blister pack.

### Nucleic Acids, Vectors, Host Cells and Methods of Production

Provided is an isolated nucleic acid molecule encoding an antibody molecule or conjugate of the invention. Nucleic acid molecules may comprise DNA and/or RNA and may be partially or wholly synthetic.

An isolated nucleic acid molecule may be used to express an antibody molecule or conjugate of the invention. The nucleic acid will generally be provided in the form of an expression vector. Another aspect of the invention thus provides an expression vector comprising a nucleic acid as described above. Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the nucleic acid in a host cell. Vectors may be plasmids, viral e.g. phage, or phagemid, as appropriate in the context.

A nucleic acid molecule or expression vector as described herein may be introduced into a host cell. Techniques for the introduction of nucleic acid or vectors into host cells are well established in the art and any suitable technique may be employed. A range of host cells suitable for the production of recombinant antibody molecules and conjugates are known in the art, and include bacterial, yeast, insect or mammalian host cells. A preferred host cell is a mammalian cell, such as a CHO, NS0, or HEK cell, for example a HEK293 cell.

Another aspect of the invention provides a method of producing an antibody molecule, or conjugate, comprising expressing a nucleic acid encoding the antibody molecule, or conjugate, in a host cell and optionally isolating and/or purifying the antibody molecule, or conjugate, thus produced. Methods for culturing host cells are well-known in the art. The method may further comprise isolating and/or purifying the antibody molecule or conjugate. Techniques for the purification of recombinant antibody molecules, or conjugates, are well-known in the art and include, for example HPLC, FPLC, or affinity chromatography, e.g. using Protein A or Protein L. In some embodiments, purification may be performed using an affinity tag on antibody molecule. The method may also comprise formulating the antibody molecule, or conjugate, into a pharmaceutical composition, optionally with a pharmaceutically acceptable excipient or other substance as described herein.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1—Cloning of Fibroblast Activation Protein (FAP) Including Characterization, Phage Display Selection Against Antigen and Isolation of C5 and 7NP2 Antibodies in scFv Format

### 1.1 Expression Procedure

A human FAP-ECD (extracellular domain) recombinant fragment containing a C-terminal His6 tag was expressed using transient gene expression (TGE) in CHO—S cells. For 1 mL of production 4×10⁶ CHO—S cells in suspension were centrifuged and resuspended in 1 mL of a suitable medium. 0.9 μg of plasmid DNA followed by 2.5 μg polyethylene imine (PEI; 1 mg/mL solution in water at pH 7.0) per million cells were then added to the cells and gently mixed. The transfected culture was incubated in a shaker incubator at 31° C. for 6 days. The protein fragment was purified from the cell culture medium by using nickel affinity chromatography and then dialyzed into HEPES buffer (100 mM NaCl, 50 mM HEPES, pH 7.4) and stored at −80° C.

### 1.2 Antigen Characterization

The human FAP-ECD was analyzed by SDS-PAGE and by size exclusion chromatography using a Superdex 200 increase 10/300 GL column on an ÄKTA FPLC. The enzymatic activity of the human FAP-ECD on the Z-Gly-Pro-AMC substrate was measured at room temperature on a microtiter plate reader, monitoring the fluorescence at an excitation wavelength of 360 nm and an emission wavelength of 465 nm. The reaction mixture contained substrate (25 μM), protein (70 nM, constant), assay buffer (50 mM Tris, 100 mM NaCl, 1 mM EDTA, pH=7.4).

### 1.3 Antigen Biotinylation

The purified human FAP-ECD recombinant fragment was randomly biotinylated with N-hydroxysuccinimide (NHS) ester-activated biotins. The biotin-labelling reaction was carried with an 80× molar excess of NHS-biotin on Hula Shaker for 1 h at room temperature. The reaction was then quenched with Tris-HCl (pH:7.4), the biotin-labelled protein was loaded on a pre-equilibrated PD10 column and dialyzed into HEPES buffer (100 mM NaCl, 50 mM HEPES, pH 7.4) overnight at 4° C.

### 1.4 Phage Display Selection

The biotinylated human FAP-ECD was used to perform biopanning with Dynabeads. Briefly, the biotinylated human FAP-ECD (final concentration 120 pmol) was incubated with 800 μL of a pre-blocked phage display library for 30 minutes. After several washes with HEPES buffer (100 mM NaCl, 50 mM HEPES, pH 7.4), selected phages were eluted by reducing the disulphide bonds in the biotin linker with triethylamine. Isolated phages were then amplified in E. coli strain TG-1 and precipitated from the supernatant with polyethylene glycol.

After two rounds of biopanning, clones were screened by ELISA. Avidin-coated ELISA plates were incubated with biotinylated human FAP-ECD. The supernatants of selected induced monoclonal clones of the E. coli TG-1 cultures expressing scFv antibody fragments were added to the ELISA plates and bound scFvs were detected using the anti-c-myc antibody 9E10 followed by us of an anti-mouse IgG—horseradish peroxidase (HRP) conjugate.

### 1.5 In Vitro Characterization of Antibody C5 in scFv Format

The antibody clone that resulted in the highest ELISA signal, C5, was produced in E. Coli strain TG-1. A TG-1 culture was grown at 37° C. in 2×TY/100 μg/ml ampicillin. At OD600=0.5, 1 mM isopropyl-thio-galactopyranoside (IPTG) was added to induce expression of the scFv; the culture was incubated on a bacterial incubator shaking at 175 rpm at 30° C. overnight. The culture was then centrifuged, and the supernatant purified from the cell culture medium by protein-A affinity chromatography and then dialyzed against PBS and stored in PBS at −80° C. The C5 scFv was then characterized by size exclusion chromatography using a Superdex 75 increase 10/300 GL column on an ÄKTA FPLC. SDS-PAGE analysis was also performed with 4-12% Bis-Tris gel under reducing and non-reducing conditions (FIG. 1).

### 1.6 Cloning and Expression of Antibody C5 in scFv Format

The C5 scFv was cloned into a vector for mammalian expression. The primers were designed to add Nhel and Hindlll restriction sites: “Leader Seq DP47”> (SEQ ID NO: 42) (TCCTCCTGTTCCTCGTCGCTGTGGCTACAGGTGTGCACTCGGAGGTGCAGCTGTTGGAGTCTGGG) and “Hindlll Stop Myc”<(SEQ ID NO: 43) (TCGATAAGCTTTTATGCGGCCCCATTCAGATCCTCTTC) to add the restriction site for Hindlll.

The resulting fragment was PCR amplified with “Nhel_leader”> (SEQ ID NO: 44) (CTAGCTAGCGTCGACCATGGGCTGGAGCCTGATCCTCCTGTTCCTCGTCGCTGTGGC) and “Hindlll Stop Myc”<(SEQ ID NO: 43) to add the restriction site for Nhel. The PCR product was digested with Nhel and Hindlll and ligated into a vector digested with the same enzymes.

C5 scFv was then expressed using transient gene expression (TGE) in CHO—S cells (as described above). The C5 scFv was purified from the cell culture medium by protein A affinity chromatography and then dialyzed against PBS and stored in PBS at −80° C. The amino acid sequence of C5 scFv is shown in SEQ ID NO: 25.

### 1.7 Construction and Cloning of an Affinity Maturation Library

An affinity maturation library was constructed in a phagemid vector by inserting random mutations within the complementary-determining region 2 (CDR2) of the variable heavy chain and variable light chain of C5 scFv.

Primers were designed to randomize the CDR2 of the VH and VL of C5 scFv. Three different fragments were amplified with:

1) ″Lmb3long″ >
(SEQ ID NO: 45)
(CAGGAAACAGCTATGACCATGATTAC)
and
″DP47 rCDR2″ <
(SEQ ID NO: 46)
(GCCCTTCACGGAGTCTGCGTAGTATGTMNNACCACCMNNMNNMNN)
2) ″DP47 CDR2″ >
(SEQ ID NO: 47)
(ACATACTACGCAGACTCCGTGAAGGGC)
and
″DPK22 rCDR2″ <
(SEQ ID NO: 48)
(GAACCTGTCTGGGATGCCAGTMNNCCTMNNGGATGCMNNATA)
3) ″DPK22 rCDR2″ >
(SEQ ID NO: 49)
(ACTGGCATCCCAGACAGGTTCAGTG)
and
″Fdseqlong″ <
(SEQ ID NO: 50)
(GACGTTAGTAAATGAATTTTCTGTATGAGG)

Fragments 1 and 2 were PCR assembled with primers “Lmb3long”> and “DPK22 rCDR2”<.

Fragments 2 and 3 were PCR assembled using as primers “DP47 CDR2”> and “Fdseqlong”<.

The resulting two PCR fragments were assembled using primers “Lmb3long”> and “Fdseqlong”<. The PCR product was digested with Notl and Ncol and ligated into a vector digested with the same enzymes.

Using electroporation, the resulting ligation product was introduced into fresh, electrocompetent E. Coli TG-1 cells prepared by washing the cells twice in 1 mM HEPES/5% glycerol and twice with 10% glycerol in water. The cells were resuspended in 10% glycerol to a density of approximately 2×10¹¹ cells. The cells were subjected to electroporation after mixing with the purified ligation product, spread on agar plates and incubated at 37° C. overnight. The following day, cells were rescued from the plates and phage were produced by superinfection with helper phage, followed by PEG/NaCl precipitation.

### 1.8 Phage Display Selection on the Affinity Matured CDR2 Library of C5

Biopanning of the affinity maturation library was performed with biotinylated human FAP-ECD. After one round of panning (as described above), a total of 12 positive clones were identified using ELISA and analyzed for binding to human FAP by BIAcore analysis. The clone “7NP2” with the highest affinity for human FAP was then further characterized.

### 1.9 Cloning, Expression and In Vitro Characterization of the 7NP2 scFv

The 7NP2 scFv was cloned into a vector for mammalian expression employing the same cloning strategy as described above for C5 scFv. The reformatted clone was expressed using transient gene expression (TGE) in CHO—S cells as described above.

7NP2 in scFv format was analyzed using size-exclusion chromatography using a Superdex 75 increase 10/300 GL column on an ÄKTA FPLC. SDS-PAGE analysis was performed using a 4-12% Bis-Tris gel under reducing and non-reducing conditions (FIG. 2).

The amino acid sequence of 7NP2 in scFv format is shown in SEQ ID NO: 11.

### 1.10 Results

A new anti-FAP antibody termed “C5” in scFv format was isolated using phage display and characterized using SDS-PAGE (FIG. 1A) and SEC (FIG. 1B) analysis.

After the construction of a C5 CDR2 affinity matured library, the anti-FAP antibody “7NP2” in scFv format was selected based on its affinity for human FAP and characterized using SDS-PAGE and SEC analysis (FIG. 2). These results showed that the C5 and 7NP2 scFv had the expected molecular weight under reducing and non-reducing conditions and were eluted from the SEC columns at 11.42 mL and 11.53 mL, respectively. Both antibody molecules showed excellent purity, as evidenced by the single peak observed by SEC.

### Example 2: Characterization of C5 and 7NP2 scFv Antibodies

### 2.1 Biacore Analysis

Affinity measurements of the C5 and 7NP2 scFv antibodies were performed by Surface Plasmon Resonance using BIAcore X100 instrument with a biotinylated human FAP-ECD coated SA chip. A final coating of 1500 RU was achieved.

Samples were injected at a concentration of 1250 nM, 450 nM and 225 nM for the C5 scFv and of 1350 nM, 675 nM, 338 nM and 170 nM for the 7NP2 scFv. No regeneration of the SA chip was performed between each run, scFvs were left to detach from the antigen. KD measurements were assessed using the BIAevaluation 3.2 software.

### 2.2 Results

The affinity (Kd) of the “C5” antibody in scFv format against human FAP as measured by BIAcore was calculated as 130 nM (FIG. 3A). The affinity (Kd) of the affinity-matured “7NP2” antibody in scFv format affinity against human FAP as measured by BIAcore was calculated as 10 nM (FIG. 3B), representing a substantial improvement over the parent C5 scFv antibody.

### Example 3: Cloning, Expression and In Vitro Characterization of the 7NP2 Antibody in IqG1 Format

### 3.1 Cloning of the 7NP2 antibody into IgG1 format

Cloning of the 7NP2 antibody in IgG1 format commenced by cloning the light chain. Primers were designed to insert Spel and BsiWI restriction sites:

″086_LC22_1PCR″ >
(SEQ ID NO: 51)
(TCCTCCTGTTCCTCGTCGCTGTGGCTACAGGTGTGCACTCGGAAATTGTGTTGACGCAGTCTCCAGG)
and
″DPK22_BsiWI″ <
(SEQ ID NO: 52)
(GCAGCCACCGTACGTTTGATTTCCACCTTGGTCCCTTG).

The resulting fragment was PCR amplified to add the leader sequence with the following primers:

″063-L19-LC-SF″ >
(SEQ ID NO: 53)
(ATTAACTAGTGTCGACCATGGGCTGGAGCCTGATCCTCCTGTTCCTCGTCGCTGTGGC)
and
″DPK22_BsiWI″ <.
(SEQ ID NO: 52)

The resulting fragment was digested with Spel and BsiWI and ligated into a suitable vector previously digested with the same restriction enzymes.

The cloning procedure was continued with the cloning of the 7NP2 heavy chain.

Primers were designed to insert Hindlll and Xhol restriction sites:

″Leader Seq DP47″ >
(SEQ ID NO: 42)
(TCCTCCTGTTCCTCGTCGCTGTGGCTACAGGTGTGCACTCGGAGGTGCAGCTGTTGGAGTCTGGG)
and
″DP47_XhoI″ <
(SEQ ID NO: 54)
(TGGTCGACGCACTCGAGACGGTGACCAGGGT).

The fragment was PCR amplified to insert the leader sequence. The primers designed were:

″061-L19-HC″ >
(SEQ ID NO: 55)
(ATTAAAGCTTGTCGACCATGGGCTGGAGCCTGATCCTCCTGTTCC)
″DP47_XhoI″ <.
(SEQ ID NO: 54)

The final PCR product was digested with Hindlll and Xhol and ligated into a suitable vector with the light chain as insert previously digested with the same restriction enzymes. The amino acid sequence of the 7NP2 antibody in IgG1 format is shown in SEQ ID NOs 13 and 14.

The same cloning strategy was used to prepare the anti-hen egg lysozyme antibody “KSF” in IgG1 format (used herein as negative control). The amino acid sequence of the KSF antibody in IgG1 format is shown in SEQ ID NOs 28 and 29.

### 3.2 Characterization of the 7NP2 Antibody in IgG1 Format

Antibody 7NP2 in IgG1 format was expressed using transient gene expression (TGE) in CHO—S cells and purified by protein-A affinity chromatography, dialyzed and stored in PBS (as described above).

Proteins were characterized by SDS-PAGE and size-exclusion chromatography using a Superdex 200 increase 10/300 GL column on an ÄKTA FPLC as also described above (FIG. 4).

### 3.3 Immunofluorescence Analysis

Immunofluorescence experiments were performed with 7NP2 and KSF (negative control), both in IgG1 format on cryosections of renal cell carcinoma SKRC52-hFAP tumor tissue slides, which were transduced to express human FAP. The 7NP2 and KSF IgG1 molecules were conjugated to FITC to allow detection. Cryostat sections (10 μm) were stained using the 7NP2 and KSF IgG1 conjugates at a final concentration of 10 μg/mL and detected with rabbit anti-FITC and goat anti-rabbit AlexaFluor488 antibody. Slides were mounted with fluorescent mounting medium and analyzed with a microscope (FIG. 5).

### 3.4 Ex-Vivo Experiments

5×10⁶ SKRC52 renal cell carcinoma cells transduced with hFAP were implanted subcutaneously in the flank of eight-week-old female BALB/c nude mice. For ex-vivo immunofluorescence analysis, when tumors reached a size of 150-250 mm³, mice were injected with 100 μg 7NP2 IgG1-FITC or KSF IgG1-FITC and sacrificed 24 hours after injection. Organs were excised and embedded in cryo-embedding medium and cryostat section (10 μm) were stained using the following antibodies: rabbit anti-FITC and goat anti-rabbit AlexaFluor488. Slides were mounted with fluorescent mounting medium and analyzed with a microscope (FIG. 6).

### 3.5 Epitope ELISA

The epitopes bound by the anti-FAP antibodies were evaluated using ELISA through competition ELISA with known anti-FAP antibodies (F5 and ESC11; see below).

To evaluate binding, 100 nM of biotinylated human FAP were coated on Streptawell plates for 30 min at 37° C.

The C5 antibody in IgG2a format was incubated together with the 7NP2 antibody in IgG1 format, the F5 antibody in IgG1 format (WO2016/116399), the ESC11 antibody in IgG1 format (Fisher et al., (2012) Clin Cancer Res; 18(22)), or the (negative control) antibody KSF in IgG1 format for 1.5 h at room temperature at a concentration of 5 μg/mL in the antigen-coated wells. After 3 washes with PBS, binding of the antibodies to human FAP was detected using anti-human or anti-murine HRP conjugates (FIG. 7).

### 3.6 Results

The 7NP2 antibody in IgG1 format showed the expected molecular weight under reducing and non-reducing conditions when analysed by SDS-PAGE and good purity as evidenced by the single peak observed using SEC (FIG. 4).

The immunofluorescence analysis on SKRC52-hFAP tissue sections using the 7NP2 antibody in IgG1 format confirmed that the antibody is capable of binding human FAP in tumor sections (FIG. 5). The KSF antibody in IgG1 format was used as negative control and showed no binding under the same conditions (FIG. 5). The ability of the 7NP2 antibody in IgG1 format to efficiently target tumors was confirmed by through ex vivo analysis of binding of 7NP2(IgG1) to tumors expressing FAP in tumor-bearing mice (FIG. 6A). No binding was seen with the negative control KSF(IgG1) as was expected (FIG. 6B).

The results of the epitope ELISA showed that the C5 and 7NP2 antibody molecules bind the same epitope and confirmed that 7NP2 has the higher affinity for human FAP, whereas the anti-FAP F5 and ESC11 antibody molecules bound different FAP epitopes to antibody C5 (and 7NP2), as both antibodies were able to bind to FAP simultaneously. The negative control antibody KSF in IgG1 format showed no binding to human FAP, as expected (FIG. 7).

### Example 4: Cloning, Expression and In Vitro Characterization of the 7NP2 Antibody in SIP Format

### 4.1 Cloning, Expression and In Vitro Characterization of the 7NP2 Antibody in SIP Format

The 7NP2 antibody was cloned into SIP (small immunoprotein) format and the protein was expressed in CHO cells using pcDNA3.1 as the expression vector. The gene encoding the 7NP2 antibody was PCR amplified with:

″Leader Seq DP47″ >
(SEQ ID NO: 42)
and
″CH4 DPK22″ <
(SEQ ID NO: 56)
(CACGCGGGCCCCCAGAGCCTCCGGATTTGATTTCCACCTTGGTCCCTTG).
A gene containing CH4 domain was PCR amplified with:
″CH4″ >
(SEQ ID NO: 57)
(TCCGGAGGCTCTGGGGGCCCGCGTG)
and
″NotI Stop CH4″ <
(SEQ ID NO: 58)
(TTTTCCTTTTGCGGCCGCCTAGCAGCCACCCCTCCTCGATGACTC).

The resulting PCR fragments were PCR assembled and cloned into the mammalian expression vector pcDNA3.1(+) using a Nhel/Notl restriction site, as described previously.

The SIP protein was expressed using transient gene expression in CHO cells as described previously and purified from the cell culture medium to homogeneity by Protein A chromatography. The 7NP2 antibody in SIP format was analyzed by size-exclusion chromatography using a Superdex 200 increase 10/300 GL column on an ÄKTA FPLC. SDS-PAGE was performed using a 4-12% Bis-Tris gel under reducing and non-reducing conditions (FIG. 8).

The amino acid sequence of 7NP2 in SIP format is shown in SEQ ID NO: 15.

The same cloning strategy was used to prepare antibody KSF in SIP format. The amino acid sequence of KSF in SIP format is shown in SEQ ID NO: 30.

### 4.2 Flow Cytometry Analysis

Binding of the 7NP2 antibody in SIP format to cell-expressed human FAP was tested using two cell lines artificially transduced to express human FAP (hFAP), with the corresponding wild-type cell line (which does not express human FAP) acting as a negative control. The cell lines used were the human renal cell carcinoma cell line SKRC52 and the human fibrosarcoma cell line HT-1080.

Specifically, SKRC52-hFAP, SKRC52 wild-type (wt), HT-1080-hFAP and HT-1080 wt cells were detached from cell culture plates using Accutase, counted and suspended to a final concentration of 1×10⁶ cells/mL in FACS buffer (0.5% BSA, 2 mM EDTA in PBS). Cells were incubated with the 7NP2 or KSF antibodies in SIP format and binding detected using an anti-IgE antibody (25 μg/ml), followed by staining with the anti-rat AlexaFluor488 antibody. Cells were analyzed on a CytoFLEX cytometer (Beckman Coulter). The raw data were processed using the FlowJo 10.4 software.

### 4.3 Results

FIG. 8A shows the results of SDS-PAGE analysis of antibody 7NP2 in SIP format. The protein exhibited the expected size of 77 kDa under non-reducing conditions and 38 kDa under reducing conditions. FIG. 8B shows the size exclusion chromatogram of the 7NP2 antibody in SIP format. The 7NP2(SIP) showed excellent purity as evidenced by the single peak observed and was eluted from the SEC column at 14 mL.

Flow cytometry analysis using HT-1080 and SKRC52 cells (both wildtype cells and cells transduced to express human FAP, respectively) confirmed the ability of the 7NP2 antibody in SIP format to bind cell-expressed human FAP. The 7NP2 antibody in SIP format bound to FAP-expressing cells with high specificity and did not show binding when incubated with the wild-type cell line which does not express FAP. The KSF antibody in SIP format was used as negative control in this experiment and did not show binding to any of the cell lines tested, as expected (FIG. 9).

### Example 5: Cross-Reactivity of the C5 and 7NP2 Antibodies Against Human and Canine FAP

### 5.1 Cloning, Expression and Characterization of Canine FAP (caFAP) Extracellular Domain

The gene for the canine FAP (caFAP) extracellular domain (ECD) was PCR amplified using the following primers:

1) ″Nhei_leader″ >
(SEQ ID NO: 44)
(CTAGCTAGCGTCGACCATGGGCTGGAGCCTGATCCTCCTGTTCCTCGTCGCTGTGGC)
and
″caFAP_BamHI″ <
(SEQ ID NO: 59)
(CATTCTGGATCCGTTTGAGCCACTGAAGGCACACGCGCTC);
2) ″caFAP_BamHI″ >
(SEQ ID NO: 60)
(GCTCAAACGGATCCAGAATGTGAGTGTCCTGTCCATTTGC)
and
″NotI-STOP-HIS″ <
(SEQ ID NO: 61)
(TTTCCTTTTGCGGCCGCTCATTAAGCTATCAGTGATGGTGATGGTGATG).

The resulting two PCR fragments were PCR assembled and cloned into the mammalian expression vector pcDNA3.1(+) by a Nhel/Notl restriction site, as described previously.

The caFAP-ECD was expressed using transient gene expression in CHO cells as described above and purified from the cell culture medium by using nickel affinity chromatography and then dialyzed into HEPES buffer (100 mM NaCl, 50 mM HEPES, pH 7.4) and stored at −80° C.

### 5.2 ELISA Analysis

Cross-reactivity of the anti-hFAP antibodies with caFAP-ECD was tested using ELISA. 120 nM of caFAP-ECD was coated on Maxisorp plates overnight at 4° C. The anti-hFAP antibodies C5, 7NP2, F5 (WO2016/116399), ESC11 (Fisher et al., (2012) Clin Cancer Res; 18(22)), 427819 IgG (R&D system, FAB3715A), or the (negative control) KSF antibody, all in IgG format were incubated in the wells for 1.5 h at room temperature at a concentration of 5 μg/mL. Specifically, the C5 antibody was employed in IgG2a format, while all other antibodies, including the 7NP2 antibody, were in IgG1 format. The differences between the IgG2a and IgG1 formats are not expected to affect binding to FAP. After 3 washes with PBS, binding of the antibodies to caFAP-ECD was detected using an anti-human or anti-murine antibody HRP conjugate.

### 5.3 Results

The anti-hFAP 7NP2 antibody and its parental antibody C5 were able to recognize and bind to caFAP-ECD, the anti-hFAP 7NP2 antibody in particular showing strong binding. In contrast, the anti-hFAP antibodies F5, ESC11, the commercial anti-hFAP antibody 427819 and the negative control KSF IgG1 antibody did not bind to caFAP-ECD (FIG. 10). An anti-HIS tag antibody-HRP conjugate was used to confirm that coating of the wells with caFAP-ECD was performed correctly.

The cross-reactivity of the 7NP2 antibody with both human and canine FAP, allows the activity and tolerability of this antibody to be tested in canines as model organisms, that are expected to be more predictive of efficacy in human patients than mouse models. In addition, the 7NP2 antibody is expected to find application in the treatment of cancer in canines, including in domestic dogs.

### Example 6: Cloning, Expression and Characterization of the IL2-7NP2-TNF^{m} Conjugates

### 6.1 Cloning, Expression and Characterization of the IL2-7NP2-TNF^{mut} Conjugates

### 6.1.1 IL2-7NP2-TNF^{mut} Conjugate

An IL2-7NP2-TNF^{mut} fusion protein containing the antibody 7NP2 in scFv format fused to a mutated version of human TNFα (arginine to alanine mutation in the amino acid at position 108 of human TNF, corresponding to position 32 in the soluble form) at its C-terminus via a 15-amino acid linker and to human IL2 at its N-terminus via a 12-amino acid linker was prepared.

Specifically, the gene encoding the 7NP2 antibody was PCR amplified with:

″Link-F8 VH″ >
(SEQ ID NO: 62)
(GCTCTTCAGGCGGCTCTGGCGGAGCTTCCGAGGTGCAGCTGTTGGAGT)
and
″L19-SSG″ <
(SEQ ID NO: 63)
(CGGAAGAGCTACTACCCGATGAGGAAGATTTGATTTCCACCTTGGTCC).

The gene encoding human IL2 was PCR amplified with:

″NheI_leader″ >
(SEQ ID NO: 44)
(CTAGCTAGCGTCGACCATGGGCTGGAGCCTGATCCTCCTGTTCCTCGTCGCTGTGGC)
and
″hIL2-Li12aa″ <
(SEQ ID NO: 64)
(GCCAGAGCCGCCTGAAGAGCCGTCACCAGTCAGTGTTGAGATGATGC).

The fragment containing the TNF gene was PCR amplified with

″link-hsTNF″ >
(SEQ ID NO: 65)
(CGGGTAGTAGCTCTTCCGGCTCATCGTCCAGCGGCGTCAGATCATCTTCTCGAAC)
and
″NotISTOP-hsTNF″ <
(SEQ ID NO: 66)
(TTTCCTTTTGCGGCCGCTCATTAAGCTATCACAGGGCAATGATCCCAAAG).

The resulting PCR fragments were PCR assembled and cloned into the mammalian expression vector pcDNA3.1(+) using a Nhel/Notl restriction site, as described previously.

The fusion protein was expressed using transient gene expression in CHO cells as described previously and purified from the cell culture medium to homogeneity by Protein A chromatography. The IL2-7NP2-TNF^{mut} conjugate was analyzed by size-exclusion chromatography using a Superdex 200 increase 10/300 GL column on an ÄKTA FPLC. SDS-PAGE was performed using a 4-12% Bis-Tris gel under reducing and non-reducing conditions (FIGS. 11A and 11B). The amino acid sequence of the IL2-7NP2-TNF^{mut} conjugate is shown in SEQ ID NO: 31.

The same cloning strategy was used to prepare an IL2-KSF-TNF^{mut} conjugate (used as negative control). The amino acid sequence of the IL2-KSF-TNF^{mut} conjugate is shown in SEQ ID NO: 36.

### 6.1.2 IL2-7NP2-TNF^{mut} v2 conjugate

A second version (v2) of IL2-7NP2-TNF^{mut} fusion protein (IL2-7NP2-TNF^{mut} v2) containing the antibody 7NP2 in scFv format fused to the mutated version of human TNFα (arginine to alanine mutation in the amino acid at position 108 of human TNF, corresponding to position 32 in the soluble form) at its C-terminus via a 16-amino acid linker and to human IL2 at its N-terminus via a 16-amino acid linker was prepared.

Specifically, the gene encoding for human 7NP2, human IL2 and human TNF (R32A) were PCR amplified, PCR assembled and cloned into a mammalian expression vector using Nhel and Notl restriction enzymes. In a first reaction human IL2 and the VH of 7NP2 were amplified using primers

″NheI_leader″ >
(SEQ ID NO: 44)
(CTAGCTAGCGTCGACCATGGGCTGGAGCCTGATCCTCCTGTTCCTCGTCGCTGTGGC)
and
PAT2
(SEQ ID NO: 84)
(GCTTGACACTGTGACCAGTGTCCCTTGACCCCAATAGTCGAACCAAGCCA).
In asecond reaction the VL of 7NP2 and human TNF (R32A) were amplified using
primers PAT3
(SEQ ID NO: 85)
(TGGCTTGGTTCGACTATTGGGGTCAAGGGACACTGGTCACAGTGTCAAGC)
and
″NotISTOP-hsTNF″ <
(SEQ ID NO: 66)
(TTTCCTTTTGCGGCCGCTCATTAAGCTATCACAGGGCAATGATCCCAAAG).

The resulting PCR products were assembled using primers “Nhel_leader” and “NotISTOP-hsTNF”< and digested with Nhel and Notl and cloned into a mammalian expression vector, as described previously.

The fusion protein was expressed using transient gene expression (TGE) in mammalian cells. For 1 mL of production 4×10⁶ cells in suspension were centrifuged and resuspended in 1 mL of a suitable medium. 0.5 μg of plasmid DNAs followed by 2.5 μg polyethylene imine (PEI; 1 mg/mL solution in water at pH 7.0) per million cells were then added to the cells and gently mixed. The transfected cultures were incubated in a shaker incubator at 31° C. for 6 days. The fusion protein was purified from the cell culture medium by protein A affinity chromatography. The fusion protein was analyzed by size-exclusion chromatography using a Superdex 200 increase 10/300 GL column on an ÄKTA FPLC system. SDS-PAGE was performed with 10% Bis-Tris gel in MOPS buffer under reducing and non-reducing conditions (FIGS. 11C and 11D).

The amino acid sequence of the IL2-7NP2-TNF^{mut} v2 conjugate is shown in SEQ ID NO: 82.

### 6.2 Results

FIGS. 11A and 11C show the size exclusion chromatogram of IL2-7NP2-TNF^{mut} and IL2-7NP2-TNF^{mut} v2 respectively (Superdex 200 increase 10/300 GL column). The IL2-7NP2-TNF^{mut} and IL2-7NP2-TNF^{mut} v2 conjugates were produced with high purity as evidenced by the single peak observed following SEC analysis and eluted from the column at 11.10 mL and at 10.69 ml, respectively. FIGS. 11B and 11D show the results of SDS-PAGE analysis of IL2-7NP2-TNF^{mut} and IL2-7NP2-TNF^{mut} v2 conjugates respectively. The fusion proteins had the expected size of 60 kDa under non-reducing and reducing conditions, respectively.

### Example 7: Therapy on SKRC52-hFAP Tumor Bearing Mice with IL2-7NP2-TNF^{mut} Conjugate

### 7.1 Therapy with IL2-7NP2-TNF^{mut} Conjugate

5×10⁶ SKRC52 renal cell carcinoma cells transduced with human FAP (SKRC52-hFAP) were implanted subcutaneously in the flank of eight-week-old female BALB/c nude mice. Tumor volume was measured with a caliper and volume was calculated using the formula: tumor Size=(Length[mm]*Width²[mm])/2. When tumors reached a suitable volume (approx. 70-100 mm³), mice were treated using the IL2-7NP2-TNF^{mut} or IL2-KSF-TNF^{mut} fusion proteins. For this purpose, the fusion proteins were dissolved in PBS, and administered to the mice at a dose of 30 μg four times every 48 h. The results are expressed as tumor volume in mm³±SEM. For the therapy experiments n=4/5 mice/group.

### 7.2 Results

Therapy of SKRC52-hFAP tumors in nude BALB/c mice with the IL2-7NP2-TNF^{mut} fusion protein resulted in tumor growth retardation compared with the negative controls (IL2-KSF-TNF^{mut} or PBS). In one of five treated mice, a complete response to treatment with IL2-7NP2-TNF^{mut} was observed with the contribution of NK cells only, as BALB/c nude mice do not produce T cells (FIG. 12A). No notable change in body weight was detected during the therapy in SKRC52-hFAP, indicating good tolerability of the fusion protein at the dose used (FIG. 12B).

### Example 8: Cross-Reactivity of the C5 and 7NP2 Antibody with Ovine and Porcine FAP Extracellular Domain

8.1. Cloning and Expression of Ovine and Porcine FAP Extracellular Domain

The genes for the ovine FAP (oFAP) extracellular domain (ECD) and for the porcine FAP (pFAP) extracellular domain (ECD) were purchased in pcDNA 3.1(+) vector from GenScript.

oFAP-ECD and pFAP-ECD were expressed using transient gene expression in CHO cells as described above and purified from the cell culture medium by using nickel affinity chromatography and then dialyzed into HEPES buffer (100 mM NaCl, 50 mM HEPES, pH 7.4) and stored at −80° C.

### 8.2 ELISA Analysis

Cross-reactivity of the anti-hFAP antibodies with oFAP-ECD and pFAP-ECD was tested using ELISA. 120 nM of oFAP-ECD or pFAP-ECD was coated on Maxisorp plates overnight at 4° C. The anti-hFAP antibodies C5, 7NP2, F5 (WO2016/116399), ESC11 (Fisher et al., (2012) Clin Cancer Res; 18(22)), 427819 IgG (R&D system, FAB3715A), or the (negative control) KSF antibody, all in IgG format were incubated in the wells for 1.5 h at room temperature at a concentration of 5 μg/mL. Specifically, the C5 antibody was employed in IgG2a format, while all other antibodies, including the 7NP2 antibody, were in IgG1 format. The differences between the IgG2a and IgG1 formats are not expected to affect binding to FAP. After 3 washes with PBS, binding of the antibodies to oFAP-ECD and pFAP-ECD was detected using an anti-human or anti-murine antibody HRP conjugate.

### 8.3 Results

The anti-hFAP 7NP2 antibody and its parental antibody C5 were able to recognize and bind to oFAP-ECD and pFAP-ECD. In contrast, the anti-hFAP antibodies F5, ESC11, the commercial anti-hFAP antibody 427819 and the negative control KSF IgG1 antibody did not show binding to oFAP-ECD and pFAP-ECD (FIG. 13). An anti-HIS tag antibody-HRP conjugate was used to confirm that coating of the wells with oFAP-ECD or pFAP-ECD was performed correctly.

The cross-reactivity of the 7NP2 antibody with ovine and porcine FAP in addition to human and canine FAP, allows the activity and tolerability of this antibody to be tested in many different animals that are expected to be more predictive of efficacy in human patients than mouse models, notably in models of inflammatory or autoimmune diseases, such as arthritis, and of cancer.

### Example 9: Cloning, Expression and Characterization of the mIL12-7NP2 and hulL12-7NP2 Conjugates

### 9.1 Cloning, expression and characterization of the mIL12-7NP2 and hulL12-7NP2 conjugates

The fusion protein IL12-7NP2 comprises the 7NP2 antibody in single chain diabody format fused to murine IL12 or human IL12 at the N-terminus. The gene encoding 7NP2 in diabody format and the gene encoding the murine IL12 or human IL12 were PCR amplified, PCR assembled and cloned into pcDNA 3.1 (+) by Nhel/Hindlll restriction sites. The two fusion proteins were expressed using TGE in CHO—S cells, purified from the cell culture medium by protein A Sepharose affinity chromatography, dialyzed against phosphate-buffered saline (PBS) and stored in PBS at −80° C. Purified proteins were analyzed by size-exclusion chromatography using a Superdex 75 increase or 200 increase 10/300 GL column on an ÄKTA FPLC. SDS-PAGE was performed with 10% gels under reducing and non-reducing condition.

The amino acid sequences of the mIL12-7NP2 and hulL12-7NP2 conjugates are shown in SEQ ID NOs: 75 and 41 respectively.

### 9.2 Results

FIG. 14A showed that the mIL12-7NP2 conjugate had the expected molecular weight under reducing and non-reducing conditions after elution from the SEC columns. The conjugate showed excellent purity, as evidenced by the single peak observed by SEC (FIG. 14B).

FIG. 15A showed the results of SDS-PAGE analysis of the hulL12-7NP2 conjugate. The conjugate had the expected size of 110 kDa under non-reducing and reducing conditions, respectively. FIG. 15B showed the size exclusion chromatogram of hulL12-7NP2. The hulL12-7NP2 conjugate was produced with high purity as evidenced by the single peak observed following SEC analysis and eluted from the column at 11.8 mL.

### Example 10: Treatment of SKRC52-hFAP Tumor-Bearing Mice with the mIL12-7NP2 Conjugate

### 10.1 Experimental Animals

Mouse experiments were performed under a project license (license number 04/2018) granted by the Veterinäramt des Kantons Zürich, Switzerland, in compliance with the Swiss Animal Protection Act (TSchG) and the Swiss Animal Protection Ordinance (TSchV).

Fifteen Female BALB/c nude mice, aged 8 weeks with an average weight of 20 g, were used in this work and raised in a pathogen-free environment with a relative humidity of 40-60%, at a temperature between 18 and 26° C. and with daily cycles of 12 hours light/darkness according to guidelines. The animals were kept in a specific pathogen free animal facility in cages of maximum 5 mice, left for one-week acclimatization upon arrival, and subsequently handled under sterile BL2 workbenches. Specialized personnel were responsible for their feeding; food and water were provided ad libitum. Mice were monitored daily (in the morning) in weight, tumor load, appearance (coat, posture, eyes and mouth moisture) and behavior (movements, attentiveness and social behavior). Euthanasia criteria adopted were body weight loss>15% and/or ulceration of the subcutaneous tumor and/or tumor diameter>1500 mm and/or mice pain and discomfort. Mice were euthanized in CO₂ chambers.

### 10.2 Tumor Model and Therapy Study

6×10⁶ SKRC52-hFAP renal cell carcinoma cells were implanted subcutaneously in the flank of BALB/c nude mice with 0.5 ml 29 G insulin syringes. Mice were monitored daily; tumor volume was measured with a caliper and volume was calculated using the formula: tumor size=(Length[mm]*Width²[mm])/2. When tumors reached a suitable volume (approx. 100 mm³), mice were injected three times into the lateral tail vein with the pharmacological agents, mIL12-7NP2 or mIL12-KSF. mIL12-7NP2 and mIL12-KSF all dissolved in PBS (pH:7.4) and administered at a dose of 8 μg/mouse every 48 hours, three times. A saline group was included as a control.

### 10.3 Results

Therapy with mIL12-7NP2 was performed with 15 BALB/c nude mice bearing SKRC52-hFAP tumors. Mice were randomized into groups according to their tumor volume; tumor volume measurements were taken by the same experimenter to minimize any subjective bias. Treatment with the mIL12-7NP2 conjugate resulted in tumor growth retardation and tumor remission in three out of six treated mice without showing toxicity, as evidenced by the stable body weight of the mice during the treatment period (FIGS. 16A and 16D).

### Example 11: Pharmacokinetic study in a Cynomolgus Monkey

### 11.1 Non-human Primate Study

The non-human primate study was performed in accordance with the Directive 2010/63/UE of the European parliament for the protection of animals used for scientific purposes. 1 female Cynomolgus Monkey, −2 years old at the time of allocation and estimated to weigh between 2.59 and 2.66 kg was used in this study. 7NP2 in IgG1 format was administered slow bolus in peripheral veins (radial vein), using disposable needles and graduated plastic syringes, at a dose volume of 1 mL/kg body weight (which corresponds to 0.1 mg/kg of conjugate). The dose was administered to the animal on the basis of the body weight measured on the day of administration. Blood samples of ˜0.6 mL each were collected from the saphenous or cephalic vein (alternatively from other blood vessels) of the animal at approximately the following 7 time points: before dosing and at 2, 10, 20 and 30 min and 1, 2, and 4 h after treatment. Blood samples were allowed to clot in tubes for a maximum of 60 minutes at room temperature then spun down by centrifugation (10 minutes 2300 g, +4° C.). For each serum sample, 2 aliquots of 100 μL were collected in labeled secondary tubes and stored in a freezer at −80° C.

### 11.2 Pharmacokinetics Analysis

Concentrations in serum were assessed by ELISA. Briefly, 100 nM of hFAP were coated on 96 well plates overnight at 4° C. After a blocking step, serum samples were incubated for 1 h and binding detected with an anti-human IgG (Fc-specific)-Peroxidase antibody.

### 11.3 Results

The results of the pharmacokinetics (PK) study in Cynomolgus Monkey are shown in FIG. 17, which shows a logarithmic representation of the PK graphs. The PK profile was linear with a slow elimination phase as expected for the IgG format.

### Example 12: Treatment of CT26-hFAP Tumor Bearing Mice with the mIL12-7NP2 Conjugate, Alone or in Combination with an aPD-1 Checkpoint Inhibitor

### 12.1 Tumor Model and Therapy Study

5×10⁶ CT26 colon carcinoma cells transduced with hFAP (CT26-hFAP) were implanted subcutaneously in the flank of 25 eight-week-old BALB/c mice. Mice were randomized into groups according to their tumor volume; tumor volume measurements were taken by the same experimenter to minimize any subjective bias. Mice were then intravenously injected with 10 μg of mIL12-7NP2 or mIL12-KSF, starting when tumors reached approximately 100 mm³, every 48 hours for three times. In the combination group, mice received 10 μg of mIL12-7NP2 as above and 200 μg of aPD-1 checkpoint inhibitor (BioXCell cat n° BE0273) every second day for three times. All therapeutic agents were diluted in phosphate buffer saline.

### 12.2 Results

Treatment with mIL12-7NP2 (monotherapy) resulted in tumor growth retardation and tumor remission in 60% of the treated mice. When mIL12-7NP2 was used in combination with an aPD-1 checkpoint inhibitor, 100% of the treated mice showed a tumor remission stable up to 60 days. Untargeted mIL12 (mIL12-KSF) and aPD-1 (monotherapy) did not show any benefit (FIG. 18A). All treatments were very well tolerated, as evidenced by the stable body weight of the mice (FIG. 18B).

### Example 13: Pharmacokinetic Analysis of IL12-7NP2 in Cynomolgus Monkeys

### 13.1 Experimental Animals

The non-human primate study was performed in accordance with the Directive 2010/63/UE of the European parliament for the protection of animals used for scientific purposes. Twelve Cynomolgus Monkey (6 male and 6 female), ˜2 years old at the time of allocation and estimated to weigh between 2.48 and 3.28 kg were used in this study. IL12-7NP2 was administered slow bolus in peripheral veins (radial vein), using disposable needles and graduated plastic syringes, at three different dose levels (high dose group (4 monkeys) 1 mg/Kg, medium dose group (4 monkeys) 0.2 mg/Kg, low dose group (4 monkeys) 0.04 mg/Kg). The dose was administered to the animal on the basis of the body weight measured on the day of administration. Blood samples of ˜0.6 mL each were collected from the saphenous or cephalic vein (alternatively from other blood vessels) of the animal at approximately the following 7 time points: before dosing and at 10 minutes and 1, 2, 4 and 6 hours after treatment. Blood samples were allowed to clot in tubes for a maximum of 60 minutes at room temperature then spun down by centrifugation (10 minutes 2300 g, +4° C.). For each serum sample, 25 aliquots of 100 μL were collected in labelled secondary tubes and stored in a freezer at −80° C.

### 13.2 Pharmacokinetics Analysis

Quantitative measurement of IL12-7NP2 in monkey serum samples was determined by AlphaLISA bead-based immunoassay method. Briefly, AlphaLISA anti-IL12 acceptor beads were incubated with samples for 30 minutes. Biotinylated hFAP was added and let incubate with samples for 60 minutes. As last step, streptavidin donor beads were added to the solution and incubate for other 30 minutes. The luminescent/fluorescent signal resulting from an energy transfer from one bead to the other based on the capture of the molecules on the beads, was detected by EnSpire® Alpha reader.

### 13.3 Results

The results of the pharmacokinetics (PK) study are reported in FIG. 19 which shows a logarithmic representation of the PK. The PK profile was linear with a slow elimination phase at all the doses tested.

1. An antibody molecule that binds human, canine, ovine and porcine fibroblast activation protein (FAP), wherein the antibody molecule comprises a VH domain comprising a set of complementarity determining regions HCDR1, HCDR2 and HCDR3, and a VL domain comprising a set of complementarity determining regions LCDR1, LCDR2 and LCDR3, wherein:
the HCDR1, HCDR2 and HCDR3 comprise the amino acid sequences set forth in SEQ ID NOs 3, 4 and 5, respectively, and
the LCDR1, LCDR2 and LCDR3 comprise the amino acid sequences set forth in SEQ ID NOs 6, 7 and 8, respectively.

2. An antibody molecule according to claim 1, wherein the VH domain comprises the amino acid sequence set forth in SEQ ID NO: 9 and the VL domain comprises the amino acid sequence set forth in SEQ ID NO: 10.

3. An antibody molecule according to claim 1, wherein the antibody molecule comprises the heavy chain amino acid sequence set forth in SEQ ID NO: 13 or 74, and the light chain amino acid sequence set forth in SEQ ID NO: 14.

4. The antibody molecule according to claim 1, wherein the antibody molecule comprises or consists of: a single chain Fv (scFv), a small immunoprotein (SIP), a diabody, a single-chain diabody.

5. A conjugate comprising an antibody molecule according to claim 1 and a pro-inflammatory agent, an anti-inflammatory agent, a biocidal molecule, a cytotoxic molecule, or a radioisotope.

6. A conjugate according to claim 5, wherein the pro-inflammatory agent, or anti-inflammatory agent, is a cytokine.

7. A conjugate according to claim 6, wherein the antibody molecule is a single-chain diabody and is conjugated at its N-terminus to IL12, and wherein the conjugate optionally comprises the sequence set forth in SEQ ID NO: 41.

8. A conjugate according to claim 6, wherein the antibody molecule is an IgG4 molecule and the C-terminus of the heavy chain is conjugated to interferon gamma, or a variant thereof, and wherein the conjugate optionally comprises the light and heavy chain sequences set forth in SEQ ID NOs 14 and 37, respectively.

9. A conjugate according to claim 6, wherein the antibody molecule is an scFv and is conjugated at its N-terminus to interleukin-2, and at its C-terminus to tumor necrosis factor alpha (TNFα) or a variant thereof, and wherein the conjugate optionally has the sequence set forth in SEQ ID NO: 31 or 82.

10. A conjugate according to claim 6, wherein the antibody molecule is a diabody (db) and is conjugated at its C-terminus to interleukin-2, and wherein the conjugate optionally has the sequence set forth in SEQ ID NO: 76 to interleukin-2, and wherein the conjugate optionally has the sequence set forth in SEQ ID NO: 76.

11. A conjugate according to claim 6, wherein the antibody molecule is a single-chain diabody (scDb) and is conjugated at its C-terminus the sushi-domain of IL15 Receptor alpha (SD), and interleukin-15 is conjugated to the C-terminus of the sushi domain, and wherein the conjugate optionally has the sequence set forth in SEO ID NO: 79.

12. A method of treating an inflammatory disorder, autoimmune disease, or cancer in a patient, comprising administering a therapeutically effective amount of the antibody molecule of claim 1 to the patient.

13. A method of treating an inflammatory disorder, autoimmune disease, or cancer in a patient, comprising administering a therapeutically effective amount of the conjugate of claim 7 to the patient, wherein the method further comprises administering a Janus kinase (JAK) inhibitor to the patient.

14. The method according to claim 13, wherein the JAK inhibitor is selected from the group consisting of: ruxolitinib, baricitinib, tofacitinib, fedratinib, momelotinib, pacritinib, fligotinib, upadacitinib, itacitinib, decernotinib, peficitinib, deucravacitinib, abrocitinib, NDI-031301, and ritlecitinib.

15. (canceled)

16. A method of cancer in a patient, comprising administering a therapeutically effective amount of the conjugate of claim 5 to the patient, wherein the method further comprises administering an immunomodulatory agent to the patient.

17. The method according to claim 16, wherein the immunomodulatory agent is selected from the group consisting of: an anti-PD-1 antibody, anti-PD-L1 antibody, anti-LAG-3 antibody, anti-TIGIT antibody, and anti-TIM-3 antibody.

18. (canceled)

19. The method according to any one of claims 13, wherein the conjugate comprises, or consist of, the sequence set forth in SEQ ID NO: 41.

20. A nucleic acid molecule or expression vector encoding an antibody molecule or conjugate according to claim 1.

21. A host cell comprising the nucleic acid or vector of claim 20.

22. A method of producing an antibody molecule or conjugate according to claim 1, comprising culturing the host cell of claim 21 under conditions for expression of the antibody molecule or conjugate, and optionally further isolating and/or purifying the antibody molecule or conjugate following expression.


SEQUENCE LISTING (85 sequences)

SEQ ID NO: 1 - protein, 735 aa, Artificial Sequence
  human extracellular domain of Fibroblast Activation Protein (hFAP)
LRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 2 - protein, 735 aa, Artificial Sequence
  canine extracellular domain of Fibroblast Activation Protein (caFAP)
LRPSRVHDSEGGTTRALTLEDILNGTFTYKTFFPNWISGQEYLHQSTDNDIVYYNIETGESYTILSNATMKSVNASNYGLSPDRQFAYLESDYSKLWRYSYTATYHIYNLNNGEFIRRNELPRPIQYLCWSPVGSKLAYVYQNNIYLKQRPEDPPFQITYNGRENKIFNGIPDWVYEEEMLATKHALWWSPNGKFLAYAEFNDTEIPVIAYSYYGDEQYPRTINIPYPKAGAKNPVVRIFIIDTTYPQQTGPREVPVPAMIASSDYYFSWLTWVTDERVCLQWLKRIQNVSVLSICDFREGWQTWDCPKAQEHIEESRTGWAGGFFVSTPVFSYDAISYYKIFSDKDGYKHIHYIKDTVENAIQITSGKWEAINIFRVTQDSLFYSSNEFEDYPGRRNIYRISIGSSPPSKKCITCHLRKERCQYYTASFSDYAKYYALICYGPGLPISTLHDGHTDQEIKILEENKELENALKNIQLPKEEIKKLEVDDITLWYKMMLPPRFDRSKKYPLLIQVYGGPCSQSVKSVFSINWISYLASKEGIVIALVDGRGTAYQGDKLLYAVYRKLGVYEVEDQITAVRKFIEMGFIDEKRIAIWGWSYGGYVSSLALASGTGLFKCGIAVAPVSSWEYYASIYTERFMGLPTKNDNLEHYKNSTVMARAEYFRNVDYLLIHGTADDNVHFQNSAQIAKALVNAQVDFQAMWYSDQNHGIPGLSSKHLYTRMTHFLKQCFSLSD

SEQ ID NO: 3 - protein, 10 aa, Artificial Sequence
  7NP2 CDR1 VH
GFTFSSYAMS

SEQ ID NO: 4 - protein, 17 aa, Artificial Sequence
  7NP2 CDR2 VH
AIGSVGGPTYYADSVKG

SEQ ID NO: 5 - protein, 7 aa, Artificial Sequence
  7NP2 CDR3 VH
RLAWFDY

SEQ ID NO: 6 - protein, 12 aa, Artificial Sequence
  7NP2 CDR1 VL
RASQSVSSSYLA

SEQ ID NO: 7 - protein, 7 aa, Artificial Sequence
  7NP2 CDR2 VL
GASSRAT

SEQ ID NO: 8 - protein, 9 aa, Artificial Sequence
  7NP2 CDR3 VL
QQSGKGPLT

SEQ ID NO: 9 - protein, 116 aa, Artificial Sequence
  7NP2 VH domain
EVQLLESGGGLVQPGGSLRLSCAASGFTFSSYAMSWVRQAPGKGLEWVSAIGSVGGPTYYADSVKGRFTISRDNSKNTLYLQMNSLRAEDTAVYYCAKRLAWFDYWGQGTLVTVSS

SEQ ID NO: 10 - protein, 108 aa, Artificial Sequence
  7NP2 VL domain
EIVLTQSPGTLSLSPGERATLSCRASQSVSSSYLAWYQQKPGQAPRLLIYGASSRATGIPDRFSGSGSGTDFTLTISRLEPEDFAVYYCQQSGKGPLTFGQGTKVEIK

SEQ ID NO: 11 - protein, 238 aa, Artificial Sequence
  7NP2 antibody molecule in scFv format
EVQLLESGGGLVQPGGSLRLSCAASGFTFSSYAMSWVRQAPGKGLEWVSAIGSVGGPTYYADSVKGRFTISRDNSKNTLYLQMNSLRAEDTAVYYCAKRLAWFDYWGQGTLVTVSSGGGGSGGGGSGGGGEIVLTQSPGTLSLSPGERATLSCRASQSVSSSYLAWYQQKPGQAPRLLIYGASSRATGIPDRFSGSGSGTDFTLTISRLEPEDFAVYYCQQSGKGPLTFGQGTKVEIK

SEQ ID NO: 12 - protein, 14 aa, Artificial Sequence
  Linker between VH and VL in 7NP2 scFv and C5 scFv
GGGGSGGGGSGGGG

SEQ ID NO: 13 - protein, 446 aa, Artificial Sequence
  7NP2 heavy chain in IgG1 format
EVQLLESGGGLVQPGGSLRLSCAASGFTFSSYAMSWVRQAPGKGLEWVSAIGSVGGPTYYADSVKGRFTISRDNSKNTLYLQMNSLRAEDTAVYYCAKRLAWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 215 aa, Artificial Sequence
  7NP2 light chain in IgG (e.g. IgG1) format
EIVLTQSPGTLSLSPGERATLSCRASQSVSSSYLAWYQQKPGQAPRLLIYGASSRATGIPDRFSGSGSGTDFTLTISRLEPEDFAVYYCQQSGKGPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 359 aa, Artificial Sequence
  7NP2 antibody molecule in SIP format
EVQLLESGGGLVQPGGSLRLSCAASGFTFSSYAMSWVRQAPGKGLEWVSAIGSVGGPTYYADSVKGRFTISRDNSKNTLYLQMNSLRAEDTAVYYCAKRLAWFDYWGQGTLVTVSSGGGGSGGGGSGGGGEIVLTQSPGTLSLSPGERATLSCRASQSVSSSYLAWYQQKPGQAPRLLIYGASSRATGIPDRFSGSGSGTDFTLTISRLEPEDFAVYYCQQSGKGPLTFGQGTKVEIKSGGSGGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPESSRRGGC

SEQ ID NO: 16 - protein, 6 aa, Artificial Sequence
  Linker between scFv and epsilon S2_CH4 domain in the SIP format
SGGSGG

SEQ ID NO: 17 - protein, 10 aa, Artificial Sequence
  C5 CDR1 VH
GFTFSSYAMS

SEQ ID NO: 18 - protein, 17 aa, Artificial Sequence
  C5 CDR2 VH
AIDGSGGSTYYADSVKG

SEQ ID NO: 19 - protein, 7 aa, Artificial Sequence
  C5 CDR3 VH
RLAWFDY

SEQ ID NO: 20 - protein, 12 aa, Artificial Sequence
  C5 CDR1 VL
RASQSVSSSYLA

SEQ ID NO: 21 - protein, 7 aa, Artificial Sequence
  C5 CDR2 VL
GASSRAT

SEQ ID NO: 22 - protein, 9 aa, Artificial Sequence
  C5 CDR3 VL
QQSGKGPLT

SEQ ID NO: 23 - protein, 116 aa, Artificial Sequence
  C5 VH domain
EVQLLESGGGLVQPGGSLRLSCAASGFTFSSYAMSWVRQAPGKGLEWVSAIDGSGGSTYYADSVKGRFTISRDNSKNTLYLQMNSLRAEDTAVYYCAKRLAWFDYWGQGTLVTVSS

SEQ ID NO: 24 - protein, 108 aa, Artificial Sequence; identical to SEQ ID NO: 10
  C5 VL domain

SEQ ID NO: 25 - protein, 238 aa, Artificial Sequence
  C5 antibody molecule in scFv format
EVQLLESGGGLVQPGGSLRLSCAASGFTFSSYAMSWVRQAPGKGLEWVSAIDGSGGSTYYADSVKGRFTISRDNSKNTLYLQMNSLRAEDTAVYYCAKRLAWFDYWGQGTLVTVSSGGGGSGGGGSGGGGEIVLTQSPGTLSLSPGERATLSCRASQSVSSSYLAWYQQKPGQAPRLLIYGASSRATGIPDRFSGSGSGTDFTLTISRLEPEDFAVYYCQQSGKGPLTFGQGTKVEIK

SEQ ID NO: 26 - protein, 446 aa, Artificial Sequence
  C5 heavy chain in murine IgG2a format
EVQLLESGGGLVQPGGSLRLSCAASGFTFSSYAMSWVRQAPGKGLEWVSAIDGSGGSTYYADSVKGRFTISRDNSKNTLYLQMNSLRAEDTAVYYCAKRLAWFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 27 - protein, 215 aa, Artificial Sequence
  C5 light chain in murine IgG2a format
EIVLTQSPGTLSLSPGERATLSCRASQSVSSSYLAWYQQKPGQAPRLLIYGASSRATGIPDRFSGSGSGTDFTLTISRLEPEDFAVYYCQQSGKGPLTFGQGTKVEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 28 - protein, 448 aa, Artificial Sequence
  KSF heavy chain in IgG1 format
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPKVSLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 214 aa, Artificial Sequence
  KSF light chain in IgG1 format
SSELTQDPAVSVALGQTVRITCQGDSLRSYYASWYQQKPGQAPVLVIYGKNNRPSGIPDRFSGSSSGNTASLTITGAQAEDEADYYCQSSPLNRLAVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 30 - protein, 362 aa, Artificial Sequence
  KSF antibody molecule in SIP format
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPKVSLFDYWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSSPLNRLAVVFGGGTKLTVLGSGGSGGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPESSRRGGC

SEQ ID NO: 31 - protein, 555 aa, Artificial Sequence
  IL2-7NP2-TNF alpha mut conjugate
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGDGSSGGSGGASEVQLLESGGGLVQPGGSLRLSCAASGFTFSSYAMSWVRQAPGKGLEWVSAIGSVGGPTYYADSVKGRFTISRDNSKNTLYLQMNSLRAEDTAVYYCAKRLAWFDYWGQGTLVTVSSGGGGSGGGGSGGGGEIVLTQSPGTLSLSPGERATLSCRASQSVSSSYLAWYQQKPGQAPRLLIYGASSRATGIPDRFSGSGSGTDFTLTISRLEPEDFAVYYCQQSGKGPLTFGQGTKVEIKSSSSGSSSSGSSSSGVRSSSRTPSDKPVAHVVANPQAEGQLQWLNRAANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 32 - protein, 12 aa, Artificial Sequence
  Linker between IL2 and 7NP2
GDGSSGGSGGAS

SEQ ID NO: 33 - protein, 15 aa, Artificial Sequence
  Linker between 7NP2 and TNF alpha mut
SSSSGSSSSGSSSSG

SEQ ID NO: 34 - protein, 133 aa, Artificial Sequence [sequence omitted]

SEQ ID NO: 35 - protein, 157 aa, Artificial Sequence
  Soluble form of the extracellular domain of human TNFalpha (R32A) mutant
VRSSSRTPSDKPVAHVVANPQAEGQLQWLNRAANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 36 - protein, 558 aa, Artificial Sequence
  IL2-KSF-TNFalpha mut conjugate
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGDGSSGGSGGASEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPKVSLFDYWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSSPLNRLAVVFGGGTKLTVLGSSSSGSSSSGSSSSGVRSSSRTPSDKPVAHVVANPQAEGQLQWLNRAANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 37 - protein, 574 aa, Artificial Sequence
  7NP2 Heavy chain IgG4-INF gamma Mut KRG conjugate
EVQLLESGGGLVQPGGSLRLSCAASGFTFSSYAMSWVRQAPGKGLEWVSAIGSVGGPTYYADSVKGRFTISRDNSKNTLYLQMNSLRAEDTAVYYCAKRLAWFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRG

SEQ ID NO: 38 - protein, 229 aa, Artificial Sequence
  7NP2 antibody in diabody format (Db)
EVQLLESGGGLVQPGGSLRLSCAASGFTFSSYAMSWVRQAPGKGLEWVSAIGSVGGPTYYADSVKGRFTISRDNSKNTLYLQMNSLRAEDTAVYYCAKRLAWFDYWGQGTLVTVSSGGSGGEIVLTQSPGTLSLSPGERATLSCRASQSVSSSYLAWYQQKPGQAPRLLIYGASSRATGIPDRFSGSGSGTDFTLTISRLEPEDFAVYYCQQSGKGPLTFGQGTKVEIK

SEQ ID NO: 39 - protein, 473 aa, Artificial Sequence
  7NP2 antibody in single-chain diabody format (scDb)
EVQLLESGGGLVQPGGSLRLSCAASGFTFSSYAMSWVRQAPGKGLEWVSAIGSVGGPTYYADSVKGRFTISRDNSKNTLYLQMNSLRAEDTAVYYCAKRLAWFDYWGQGTLVTVSSGGSGGEIVLTQSPGTLSLSPGERATLSCRASQSVSSSYLAWYQQKPGQAPRLLIYGASSRATGIPDRFSGSGSGTDFTLTISRLEPEDFAVYYCQQSGKGPLTFGQGTKVEIKDIGGGSGGGGSGGGGEVQLLESGGGLVQPGGSLRLSCAASGFTFSSYAMSWVRQAPGKGLEWVSAIGSVGGPTYYADSVKGRFTISRDNSKNTLYLQMNSLRAEDTAVYYCAKRLAWFDYWGQGTLVTVSSGGSGGEIVLTQSPGTLSLSPGERATLSCRASQSVSSSYLAWYQQKPGQAPRLLIYGASSRATGIPDRFSGSGSGTDFTLTISRLEPEDFAVYYCQQSGKGPLTFGQGTKVEIK

SEQ ID NO: 40 - protein, 5 aa, Artificial Sequence
  Linker between VH and VL in the 7NP2 Db and 7NP2 scDb
GGSGG

SEQ ID NO: 41 - protein, 1006 aa, Artificial Sequence
  IL12-7NP2 conjugate
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSEVQLLESGGGLVQPGGSLRLSCAASGFTFSSYAMSWVRQAPGKGLEWVSAIGSVGGPTYYADSVKGRFTISRDNSKNTLYLQMNSLRAEDTAVYYCAKRLAWFDYWGQGTLVTVSSGGSGGEIVLTQSPGTLSLSPGERATLSCRASQSVSSSYLAWYQQKPGQAPRLLIYGASSRATGIPDRFSGSGSGTDFTLTISRLEPEDFAVYYCQQSGKGPLTFGQGTKVEIKDIGGGSGGGGSGGGGEVQLLESGGGLVQPGGSLRLSCAASGFTFSSYAMSWVRQAPGKGLEWVSAIGSVGGPTYYADSVKGRFTISRDNSKNTLYLQMNSLRAEDTAVYYCAKRLAWFDYWGQGTLVTVSSGGSGGEIVLTQSPGTLSLSPGERATLSCRASQSVSSSYLAWYQQKPGQAPRLLIYGASSRATGIPDRFSGSGSGTDFTLTISRLEPEDFAVYYCQQSGKGPLTFGQGTKVEIK

SEQ ID NO: 42 - DNA, 65 bp, Artificial Sequence
  “Leader Seq DP47” > primer
tcctcctgttcctcgtcgctgtggctacaggtgtgcactcggaggtgcagctgttggagtctggg

SEQ ID NO: 43 - DNA, 38 bp, Artificial Sequence
  “HindIII Stop Myc” < primer
tcgataagcttttatgcggccccattcagatcctcttc

SEQ ID NO: 44 - DNA, 57 bp, Artificial Sequence
  “NheI_leader” > primer
ctagctagcgtcgaccatgggctggagcctgatcctcctgttcctcgtcgctgtggc

SEQ ID NO: 45 - DNA, 26 bp, Artificial Sequence
  “Lmb3long” > primer
caggaaacagctatgaccatgattac

SEQ ID NO: 46 - DNA, 45 bp, Artificial Sequence
  “DP47 rCDR2” < primer
  misc_feature (29)..(30): n is a, c, g, or t
  misc_feature (38)..(39): n is a, c, g, or t
  misc_feature (41)..(42): n is a, c, g, or t
  misc_feature (44)..(45): n is a, c, g, or t
gcccttcacggagtctgcgtagtatgtmnnaccaccmnnmnnmnn

SEQ ID NO: 47 - DNA, 27 bp, Artificial Sequence
  “DP47 CDR2” > primer
acatactacgcagactccgtgaagggc

SEQ ID NO: 48 - DNA, 42 bp, Artificial Sequence
  “DPK22 rCDR2” < primer
  misc_feature (23)..(24): n is a, c, g, or t
  misc_feature (29)..(30): n is a, c, g, or t
  misc_feature (38)..(39): n is a, c, g, or t
gaacctgtctgggatgccagtmnncctmnnggatgcmnnata

SEQ ID NO: 49 - DNA, 25 bp, Artificial Sequence
  “DPK22 rCDR2” > primer
actggcatcccagacaggttcagtg

SEQ ID NO: 50 - DNA, 30 bp, Artificial Sequence
  “Fdseqlong” < primer
gacgttagtaaatgaattttctgtatgagg

SEQ ID NO: 51 - DNA, 67 bp, Artificial Sequence
  “086_LC22_1PCR”> primer
tcctcctgttcctcgtcgctgtggctacaggtgtgcactcggaaattgtgttgacgcagtctccagg

SEQ ID NO: 52 - DNA, 38 bp, Artificial Sequence
  “DPK22_BsiWI” <  primer
gcagccaccgtacgtttgatttccaccttggtcccttg

SEQ ID NO: 53 - DNA, 58 bp, Artificial Sequence
  “063-L19-LC-SF” > primer
attaactagtgtcgaccatgggctggagcctgatcctcctgttcctcgtcgctgtggc

SEQ ID NO: 54 - DNA, 31 bp, Artificial Sequence
  “DP47_XhoI” < primer
tggtcgacgcactcgagacggtgaccagggt

SEQ ID NO: 55 - DNA, 45 bp, Artificial Sequence
  “061-L19-HC” > primer
attaaagcttgtcgaccatgggctggagcctgatcctcctgttcc

SEQ ID NO: 56 - DNA, 49 bp, Artificial Sequence
  “CH4 DPK22” < primer
cacgcgggcccccagagcctccggatttgatttccaccttggtcccttg

SEQ ID NO: 57 - DNA, 25 bp, Artificial Sequence
  “CH4” > primer
tccggaggctctgggggcccgcgtg

SEQ ID NO: 58 - DNA, 45 bp, Artificial Sequence
  “NOTI STOP CH4” < primer
ttttccttttgcggccgcctagcagccacccctcctcgatgactc

SEQ ID NO: 59 - DNA, 40 bp, Artificial Sequence
  “caFAP_BamHI” < primer
cattctggatccgtttgagccactgaaggcacacgcgctc

SEQ ID NO: 60 - DNA, 40 bp, Artificial Sequence
  “caFAP_BamHI” > primer
gctcaaacggatccagaatgtgagtgtcctgtccatttgc

SEQ ID NO: 61 - DNA, 49 bp, Artificial Sequence
  “NotI-STOP-HIS” < primer
tttccttttgcggccgctcattaagctatcagtgatggtgatggtgatg

SEQ ID NO: 62 - DNA, 48 bp, Artificial Sequence
  “Link-F8 VH” > primer
gctcttcaggcggctctggcggagcttccgaggtgcagctgttggagt

SEQ ID NO: 63 - DNA, 48 bp, Artificial Sequence
  “L19-SSG” < primer
cggaagagctactacccgatgaggaagatttgatttccaccttggtcc

SEQ ID NO: 64 - DNA, 47 bp, Artificial Sequence
  “hIL2-Li12aa” < primer
gccagagccgcctgaagagccgtcaccagtcagtgttgagatgatgc

SEQ ID NO: 65 - DNA, 55 bp, Artificial Sequence
  “link-hsTNF” > primer
cgggtagtagctcttccggctcatcgtccagcggcgtcagatcatcttctcgaac

SEQ ID NO: 66 - DNA, 50 bp, Artificial Sequence
  “NotISTOP-hsTNF” < primer
tttccttttgcggccgctcattaagctatcacagggcaatgatcccaaag

SEQ ID NO: 67 - DNA, 62 bp, Artificial Sequence
  “085-LC16-1PCR” > primer
tcctcctgttcctcgtcgctgtggctacaggtgtgcactcgtcgtctgagctgactcaggac

SEQ ID NO: 68 - DNA, 42 bp, Artificial Sequence
  “DPL16_HpaI” < primer
agggggcagccttgggctggcctaggacggttaacttggtcc

SEQ ID NO: 69 - protein, 15 aa, Artificial Sequence
  Flexible linker connecting the two sets of VH and VL domains in the 7NP2 scDb
DIGGGSGGGGSGGGG

SEQ ID NO: 70 - protein, 518 aa, Artificial Sequence
  single-chain human IL12
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 71 - protein, 130 aa, Artificial Sequence
  IFN gamma Mut
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRG

SEQ ID NO: 72 - protein, 735 aa, Artificial Sequence
  ovine extracellular domain of Fibroblast Activation Protein (oFAP)
LRPSRVHNSEDSATRALTLKDILNGTFSYKTFFPNWISGQEYLHQSTDNNVVFYNIETGESYTILSNTTMKSVNASNYGLSPDRQFAYLESDYSKLWRYSYTATYHIYDLTNGEFIRRNELPRPIQYLCWSPVGSKLAYVYQNNIYLKQRPEDPPFQITYNGKENKIFNGIPDWVYEEEMLATKYALWWSPNGKFLAYAEFNDTEIPVIAYSYYGDEQYPRTINIPYPKAGAKNPVVRIFIIDATYPEHIGPREVPVPAMIASSDYYFSWLTWVTDDRICLQWLKRIQNVSVLSICDFREDWHTWNCPKTQEHIEESRTGWAGGFFVSTPVFSHDTISYYKIFSDKDGYKHIHYIKDTVENAIQITSGKWEAINIFRVTQDSLFYSSNEFEGYPGRRNIYRISIGSHSPSKKCITCHLRKKRCQYYTASFSDYAKYYALVCYGPGLPISTLHDGRTDQEIKILEDNKELENALKNIQLPKEEIKKLKVDDITLWYKMILPPQFDKSKKYPLLIQVYGGPCSQSVRSVFAVSWISYLASKEGIVIALVDGRGTAFQGDRLLYAVYRKLGVYEVEDQITAVRKFIEMGFIDEKRIAIWGWSYGGYVSSLALASGTGLFKCGIAVAPVSSWEYYASIYTERFMGLPTKDDNLKHYKNSTVMARAEYFRNVDYLLIHGTADDNVHFQNSAQIAKALVNAQVDFQAMWYSDQNHGLSGLSTKHLYIHITHFLKQCFSLSD

SEQ ID NO: 73 - protein, 709 aa, Artificial Sequence
  porcine extracellular domain of Fibroblast Activation Protein (pFAP)
IKTRALTLKDILNGTFSYKTFFPNWISGQEYLHQSTDDNVIFYNIETGESYTILSNTTMVCIQHCLPQKPLPFIYLFIYFLYFFSSREFITRNELPRPIQYLCWSPVGSKLAYVYQNNIYLKQRPEDPPFQITYNGKENKIFNGIPDWVYEEEMLATKYALWWSPNGRFLAYAEFNDTEIPVIAYSYYGDEQYPRTINIPYPKAGAKNPFVRIFIIDTSYPGHVGPREVPVPAMIASSDYYFSWFTWVTDDRICLQWLKRIQNVSVLSICDFREDWQTWNCPKTQEHIEESRTGWAGGFFVSTPVFSYDAISYYKIFSDKDGYKHIHYIKDSVENAIQITSGKWEAINIFRVTQDSLFYSSNEFEGYPGRRNIYRISIGSHPPSKKCVTCHLREKRCQYYTASFSDYAKYYALVCYGPGLPISTLHDGRTDQEIKILEENKDLEYALKNIRLPKEEIKKLDVDDITLWYKMILPPQFDRSKKYPLLIQVYGGPCSQSVRSVFSIRWISYLASKEGIVIALVDGRGTAFQGDKLLYAVYRKLGVYEVEDQITAVRKFIEMGFIDEKRIAIWGWSYGGYVSSLALASGTGLFKCGIAVAPVSSWEYYASIYTERFMGLPTKDDNLEHYKNSTVMARAEYFRNVDYLLIHGTADDNVHFQNSAQIAKALVNAQVDFQAMWYSDQNHGISGLSTKHLYTHMTHFLKQCFSLPD

SEQ ID NO: 74 - protein, 443 aa, Artificial Sequence
  7NP2 heavy chain in IgG4 format
EVQLLESGGGLVQPGGSLRLSCAASGFTFSSYAMSWVRQAPGKGLEWVSAIGSVGGPTYYADSVKGRFTISRDNSKNTLYLQMNSLRAEDTAVYYCAKRLAWFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 75 - protein, 1009 aa, Artificial Sequence
  mIL12-7NP2 conjugate
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMRPLKNSQVEVSWEYPDSWSTPHSYFSLKFFVRIQRKKEKMKETEEGCNQKGAFLVERTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSEVQLLESGGGLVQPGGSLRLSCAASGFTFSSYAMSWVRQAPGKGLEWVSAIGSVGGPTYYADSVKGRFTISRDNSKNTLYLQMNSLRAEDTAVYYCAKRLAWFDYWGQGTLVTVSSGGSGGEIVLTQSPGTLSLSPGERATLSCRASQSVSSSYLAWYQQKPGQAPRLLIYGASSRATGIPDRFSGSGSGTDFTLTISRLEPEDFAVYYCQQSGKGPLTFGQGTKVEIKDIGGGSGGGGSGGGGEVQLLESGGGLVQPGGSLRLSCAASGFTFSSYAMSWVRQAPGKGLEWVSAIGSVGGPTYYADSVKGRFTISRDNSKNTLYLQMNSLRAEDTAVYYCAKRLAWFDYWGQGTLVTVSSGGSGGEIVLTQSPGTLSLSPGERATLSCRASQSVSSSYLAWYQQKPGQAPRLLIYGASSRATGIPDRFSGSGSGTDFTLTISRLEPEDFAVYYCQQSGKGPLTFGQGTKVEIK

SEQ ID NO: 76 - protein, 377 aa, Artificial Sequence
  7NP2(Db)-huIL2 conjugate
EVQLLESGGGLVQPGGSLRLSCAASGFTFSSYAMSWVRQAPGKGLEWVSAIGSVGGPTYYADSVKGRFTISRDNSKNTLYLQMNSLRAEDTAVYYCAKRLAWFDYWGQGTLVTVSSGGSGGEIVLTQSPGTLSLSPGERATLSCRASQSVSSSYLAWYQQKPGQAPRLLIYGASSRATGIPDRFSGSGSGTDFTLTISRLEPEDFAVYYCQQSGKGPLTFGQGTKVEIKGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 77 - protein, 65 aa, Artificial Sequence
  Sushi Domain of the IL15 Receptor alpha
ITCPPPMSVEHADIWVKSYSLYSRERYICNSGFKRKAGTSSLTECVLNKATNVAHWTTPSLKCIR

SEQ ID NO: 78 - protein, 114 aa, Artificial Sequence
  human Interleukin 15 (huIL15)
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 79 - protein, 687 aa, Artificial Sequence
  7NP2(scDb)-SD-huIL15 conjugate
EVQLLESGGGLVQPGGSLRLSCAASGFTFSSYAMSWVRQAPGKGLEWVSAIGSVGGPTYYADSVKGRFTISRDNSKNTLYLQMNSLRAEDTAVYYCAKRLAWFDYWGQGTLVTVSSGGSGGEIVLTQSPGTLSLSPGERATLSCRASQSVSSSYLAWYQQKPGQAPRLLIYGASSRATGIPDRFSGSGSGTDFTLTISRLEPEDFAVYYCQQSGKGPLTFGQGTKVEIKDIGGGSGGGGSGGGGEVQLLESGGGLVQPGGSLRLSCAASGFTFSSYAMSWVRQAPGKGLEWVSAIGSVGGPTYYADSVKGRFTISRDNSKNTLYLQMNSLRAEDTAVYYCAKRLAWFDYWGQGTLVTVSSGGSGGEIVLTQSPGTLSLSPGERATLSCRASQSVSSSYLAWYQQKPGQAPRLLIYGASSRATGIPDRFSGSGSGTDFTLTISRLEPEDFAVYYCQQSGKGPLTFGQGTKVEIKGGGGSGGGGSGGGGSITCPPPMSVEHADIWVKSYSLYSRERYICNSGFKRKAGTSSLTECVLNKATNVAHWTTPSLKCIRGGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 80 - protein, 15 aa, Artificial Sequence
  linker between p40 and p35 in the single-chain IL12, between IL12 and 7NP2 VH in IL12-7NP2 conjugate, between 7NP2 VL and IL2 in 7NP2(Db)-IL2 conjugate and between 7NP2 VL and SD in 7NP2(scDb)-SD-huIL15 conjugate
GGGGSGGGGSGGGGS

SEQ ID NO: 81 - protein, 20 aa, Artificial Sequence
  linker connecting the Sushi Domain (SD) to huIL15 in the 7NP2(scDb)-SD-huIL15 conjugate
GGGSGGGGSGGGSGGGGSLQ

SEQ ID NO: 82 - protein, 562 aa, Artificial Sequence
  IL2-7NP2-TNF?mut v2 conjugate
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSKEVQLLESGGGLVQPGGSLRLSCAASGFTFSSYAMSWVRQAPGKGLEWVSAIGSVGGPTYYADSVKGRFTISRDNSKNTLYLQMNSLRAEDTAVYYCAKRLAWFDYWGQGTLVTVSSGGGGSGGGGSGGGGSKEIVLTQSPGTLSLSPGERATLSCRASQSVSSSYLAWYQQKPGQAPRLLIYGASSRATGIPDRFSGSGSGTDFTLTISRLEPEDFAVYYCQQSGKGPLTFGQGTKVEIKGGGGSGGGGSGGGGSKVRSSSRTPSDKPVAHVVANPQAEGQLQWLNRAANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 83 - protein, 16 aa, Artificial Sequence
  linker between IL2 and 7NP2, between VH and VL in 7NP2, and between 7NP2 and TNF?mut in the IL2-7NP2-TNF?mut v2 conjugate
GGGGSGGGGSGGGGSK

SEQ ID NO: 84 - DNA, 50 bp, Artificial Sequence
  PAT2 primer
gcttgacactgtgaccagtgtcccttgaccccaatagtcgaaccaagcca

SEQ ID NO: 85 - DNA, 50 bp, Artificial Sequence
  PAT3 primer
tggcttggttcgactattggggtcaagggacactggtcacagtgtcaagc